(19) United States
(12) Patent Application Publication
(10) Pub. No.: US 2023/0151069 A1
(43) Pub. Date: May 18, 2023

(54) Cyclic Peptides

(71) Applicants: LIFEARC, London (GB); Georg-August-Universität Göttingen Stiftung Öffentlichen Rechts Universitatsmedizin, Göttingen (DE)

(72) Inventors: Preeti BAKRANIA, London (GB); David MATTHEWS, London (GB); Elizabeth LOVE, London (GB); Chido MPAMHANGA, London (GB); Thomas BAYER, Göttingen (DE); Mark CARR, London (GB); Gareth HALL, London (GB); Richard COWAN, London (GB)

(21) Appl. No.: 17/905,894

(22) PCT Filed: Mar. 10, 2021
(86) PCT No.: PCT/EP2021/056039

§ 371 Date: Sep. 8, 2022

(30) Foreign Application Priority Data
Mar. 10, 2020 (GB) 2003462.5
Oct. 16, 2020 (GB) 2016449.7

(51) Int. Cl. C07K 14/47; A61K 39/00; C07K 16/18; A61P 25/28

(52) U.S. Cl. CPC C07K 14/4711; A61K 39/0007; C07K 16/18; A61P 25/28

(57) ABSTRACT

The present invention relates to cyclized peptides based on amino acids 1-14 of amyloid-beta. The cyclic peptides are useful for inducing an immune response and as vaccines for the treatment of neurodegenerative diseases such as Alzheimer's disease.

### FIELD

The present invention relates to cyclized peptides and their use as vaccines for the prevention and treatment of neurodegenerative diseases such as Alzheimer's disease.

### BACKGROUND

Alzheimer's Disease (AD) is a progressive neurodegenerative disease characterised by the presence of extracellular deposits composed of the amyloid-beta (Aβ) protein. Full-length Aβ1-42 (SEQ ID NO: 18) and Aβ1-40 (SEQ ID NO: 19), N-truncated pyroglutamate AβpE3-42 (SEQ ID NO: 20) and Aβ4-42 (SEQ ID NO: 21) are major variants of the amyloid-β protein.

Amyloid-β protein is prone to aggregate and form amyloid fibrils. Amyloid fibrils are large insoluble polymers of Aβ found in senile plaques and are a major trigger of neuron loss and dementia typical for Alzheimer's Disease. However, there is also growing evidence towards the role of soluble Aβ oligomers rather than Aβ precipitated in plaques in the development of Alzheimer's Disease. Soluble oligomers are nonfibrillar structures, which are stable in aqueous solution and remain soluble even after high speed centrifugation. Aβ plaques have shown to be poor correlates for the clinical symptomatology in AD patients, whilst soluble oligomers are suggested to be good predictors for synaptic loss [Lue L F et al, Am J Pathol 1999, 155:853-862], neurofibrillary tangles [McLean C A, et al, Ann Neurol 1999, 46:860-866] and clinical phenotype [Snowdon D A: Aging and Alzheimer's disease: lessons from the Nun Study. Gerontologist 1997, 37:150-156]. Furthermore, memory impairment and pathological changes in many AD mouse models occur well before the onset of plaque deposition [Bayer T A and, Wirths O. Front Aging Neurosci 2010, 2:1-10]. In particular it is known that Aβ tri- or tetramers are the most toxic Aβ peptides at the beginning of the pathology of AD. As such low molecular weight (LMW) oligomers of Aβ have been seen as a target in the treatment of amyloid-beta associated diseases such as AD.

Antibodies have been developed that target low molecular weight oligomers with the aim to neutralize these oligomers. Passive immunisation has been demonstrated for the antibody 9D5 detecting low molecular weight (LMW) AβpE3-42 (Wirths et al. (2010) J. Biol. Chem. 285, 41517-41524; and WO 2011/151076). The murine anti-amyloid beta (An) antibody NT4X-167 was initially raised against Aβ4-40 amyloid peptide and is reported to bind specifically to the N-truncated amyloid peptides AβpE3-42 and Aβ4-42 but not to amyloid peptide Aβ1-42 (Antonios et al Acta Neuropathol. Commun. (2013) 6 1 56). Passive immunization using NT4X-167 has been shown to be therapeutically beneficial in Alzheimer mouse models (Antonios et al Scientific Reports 5 17338; 2015, WO2013/167681). Humanised versions of NT4X useful for clinical applications, for example in the treatment of Alzheimer's disease (AD) have also been developed (WO2020/070225).

Active immunisation approaches for Alzheimer's disease have also been proposed. For example, as described in WO2006/0609718 which discloses an Aβ derived peptide, including a cyclic peptide formed via head-to-tail cyclization. The use of linear peptides based on different regions of Aβ have also been proposed, such as that described in WO2014/143087. WO2014/143087 describes an active immunisation approach targeting an N-terminal epitope of Aβ which uses a linear peptide based on Aβ as part as part of the immunogenic constructs. However, such approaches would not specifically generate antibodies to low molecular weight oligomers.

Therefore, a compound that can be used for active immunisation would be useful in the treatment of Alzheimer's Disease, in particular to target the early stages of AD development.

### SUMMARY

The present invention generally relates to specific cyclic peptides based on amino acid residues 1-14 of amyloid beta (Aβ) protein, and which preferably bind specifically to antibodies that specifically bind to low molecular weight oligomers of Aβ-protein.

Accordingly, in a first aspect, the invention relates to a cyclic peptide comprising an amino acid sequence having the sequence of formula (I) (SEQ ID NO: 1) or variant thereof:

X₁X₂X₃FX₄HDSGX₅X₆X₇X₈H (I)

wherein:
X₁ is absence or any amino acid; and
X₂ is alanine or cysteine;
X₃ is glutamic acid or cysteine;
X₄ is arginine or cysteine;
X₅ is tyrosine or cysteine;
X₆ is glutamic acid or cysteine;
X₇ is valine or cysteine; and
X₈ is histidine or cysteine
   wherein only one of X₁, X₂, X₃ and X₄ is cysteine and wherein only one of X₅, X₆, X₇ and X₈ is cysteine and the peptide is cyclized via the cysteine residue at position 1, 2, 3, or 5 and the cysteine residue at position 10, 11, 12 or 13. Preferably X₁ is present, more preferably X₁ is proline, aspartic acid, or cysteine, more preferably is cysteine or aspartic acid. Preferably there is at least 7 amino acid between the two cysteine residues present in the sequence, more preferably there is between 7 and 11, even more preferably there is 8 or 11 amino acid residues between the two cysteine residues present in the sequence.

In one embodiment the invention relates to cyclic peptides comprising the sequence of Formula (I) as described above, wherein the peptide does not comprise cysteine residues at both positions 5 and 12 or at both positions 3 and 10.

In one embodiment, X₁ is absence or any amino acid; and
a) X₁ is cysteine, X₂ is alanine, X₃ is glutamic acid, X₄ is arginine, X₅ is tyrosine, X₆ is glutamic acid, X₇ is valine and X₈ is cysteine;
b) X₂ is alanine, X₃ is cysteine, X₄ is arginine, X₅ is tyrosine, X₆ is glutamic acid, X₇ is cysteine and X₈ is histidine;
c) X₂ is cysteine, X₃ is glutamic acid, X₄ is arginine, X₅ is tyrosine, X₆ is glutamic acid, X₇ is cysteine and X₈ is histidine;
d) X₂ is cysteine, X₃ is glutamic acid, X₄ is arginine, X₅ is cysteine, X₆ is glutamic acid, X₇ is valine and X₈ is histidine;
e) X₂ is cysteine, X₃ is glutamic acid, X₄ is arginine, X₅ is tyrosine, X₆ is glutamic acid, X₇ is valine and X₈ is cysteine
f) X₂ is cysteine, X₃ is glutamic acid, X₄ is arginine, X₅ is tyrosine, X₆ is cysteine, X₇ is valine and X₈ is histidine;
g) X₂ is alanine, X₃ is cysteine, X₄ is arginine, X₅ is tyrosine, X₆ is cysteine, X₇ is valine and X₈ is histidine;
h) X₂ is alanine, X₃ is glutamic acid, X₄ is cysteine, X₅ is tyrosine, X₆ is glutamic acid, X₇ is cysteine and X₈ is histidine;
i) X₂ is alanine, X₃ is cysteine, X₄ is arginine, X₅ is cysteine, X₆ is glutamic acid, X₇ is valine and X₈ is histidine; or
j) X₂ is alanine, X₃ is cysteine, X₄ is arginine, X₅ is tyrosine, X₆ is glutamic acid, X₇ is valine and X₈ is cysteine;
   wherein the peptide is cyclized via the two cysteine residues.

In one embodiment the cyclic peptide comprises the amino acid sequence having the sequence of formula (II) (SEQ ID NO: 2) or variant thereof:

X₁ACFRHDSGYECHH (II)

wherein the peptide is cyclized via the cysteine residues located at positions 3 and 12 and wherein X₁ is as defined above. Preferably X₁ is aspartic acid.

In a further embodiment the cyclic peptide comprises an amino acid sequence or variant thereof selected from:
a) CAEFRHDSGYEVCH (SEQ ID NO:14) wherein the peptide is a cyclized via the cysteine residues located at positions 1 and 13;
b) DACFRHDSGYECHH (SEQ ID NO: 4) wherein the peptide is a cyclized via the cysteine residues located at positions 3 and 12;
c) DCEFRHDSGYECHH (SEQ ID NO: 5) wherein the peptide is a cyclized via the cysteine residues located at positions 2 and 12;
d) DCEFRHDSGCEVHH (SEQ ID NO: 10) wherein the peptide is a cyclized via the cysteine residues located at positions 2 and 10;
e) DCEFRHDSGYEVCH (SEQ ID NO: 12) wherein the peptide is a cyclized via the cysteine residues located at positions 2 and 13;
f) DCEFRHDSGYCVHH (SEQ ID NO: 9) wherein the peptide is a cyclized via the cysteine residues located at positions 2 and 11;
g) DACFRHDSGYCVHH (SEQ ID NO: 8) wherein the peptide is a cyclized via the cysteine residues located at positions 3 and 11;
h) DAEFCHDSGYECHH (SEQ ID NO: 7) wherein the peptide is a cyclized via the cysteine residues located at positions 5 and 12;
i) DACFRHDSGCEVHH (SEQ ID NO: 11) wherein the peptide is a cyclized via the cysteine residues located at positions 3 and 10; and
j) DACFRHDSGYEVCH (SEQ ID NO: 13) wherein the peptide is a cyclized via the cysteine residues located at positions 3 and 13.

Preferably the cyclic peptide comprises an amino acid sequence or variant thereof selected from:
a) DACFRHDSGYECHH (SEQ ID NO: 4) wherein the peptide is a cyclized via the cysteine residues located at positions 3 and 12;
b) DCEFRHDSGYECHH (SEQ ID NO: 5) wherein the peptide is a cyclized via the cysteine residues located at positions 2 and 12;
c) DCEFRHDSGCEVHH (SEQ ID NO: 10) wherein the peptide is a cyclized via the cysteine residues located at positions 2 and 10;
d) DCEFRHDSGYEVCH (SEQ ID NO: 12) wherein the peptide is a cyclized via the cysteine residues located at positions 2 and 13;
e) DCEFRHDSGYCVHH (SEQ ID NO: 9) wherein the peptide is a cyclized via the cysteine residues located at positions 2 and 11;
f) DACFRHDSGYCVHH (SEQ ID NO: 8) wherein the peptide is a cyclized via the cysteine residues located at positions 3 and 11; and
g) DACFRHDSGYEVCH (SEQ ID NO: 13) wherein the peptide is a cyclized via the cysteine residues located at positions 3 and 13.

In one embodiment, the cyclic peptide comprises the amino acid sequence CAEFRHDSGYEVCH (SEQ ID NO: 14) or a variant thereof, wherein the peptide is cyclized via the cysteine residues located at positions 1 and 13.

In one embodiment the cyclic peptide is cyclized via the two cysteine residues. Preferably the peptide is cyclized via a bridge having the formula —S—S— or —S—CH₂—S— between the two cysteine residues. More preferably the peptide is cyclized via a bridge having the formula —S—CH₂—S— between the two cysteine residues.

Preferably the cyclic peptide comprises the amino acid sequence CAEFRHDSGYEVCH (SEQ ID NO: 14) or variant thereof wherein the peptide is cyclized via the cysteine residues located at positions 1 and 13. More preferably the cyclic peptide comprises the amino acid sequence CAEFRHDSGYEVCH or variant thereof wherein the peptide is cyclized via a bridge having the formula —S—CH₂—S— between the two cysteine residues at positions 1 and 13.

Preferably the cyclic peptide comprises the amino acid sequence DACFRHDSGYECHH (SEQ ID NO: 4) or variant thereof wherein the peptide is cyclized via the cysteine residues located at positions 3 and 12. More preferably the cyclic peptide comprises the amino acid sequence DACFRHDSGYECHH or variant thereof wherein the peptide is cyclized via a bridge having the formula —S—CH₂—S— between the two cysteine residues at positions 3 and 12.

Preferably the cyclic peptide comprises the amino acid sequence DACFRHDSGYEVCH (SEQ ID NO: 13) or variant thereof wherein the peptide is cyclized via the cysteine residues located at positions 3 and 13. More preferably the cyclic peptide comprises the amino acid sequence DACFRHDSGYEVCH or variant thereof wherein the peptide is cyclized via a bridge having the formula —S—CH₂—S— between the two cysteine residues at positions 3 and 13.

In a further embodiment the cyclic peptide consists of the amino acid sequence CAEFRHDSGYEVCH (SEQ ID NO: 14) or variant thereof wherein the peptide is cyclized via a bridge having the formula —S—CH₂—S— between the two cysteine residues at positions 1 and 13.

In a further embodiment the cyclic peptide consists of the amino acid sequence DACFRHDSGYECHH (SEQ ID NO: 4) or variant thereof wherein the peptide is cyclized via a bridge having the formula —S—CH₂—S— between the two cysteine residues at positions 3 and 12.

In one embodiment the cyclic peptide consists of the amino acid sequence DACFRHDSGYEVCH (SEQ ID NO: 13) or variant thereof wherein the peptide is cyclized via a bridge having the formula —S—CH₂—S— between the two cysteine residues at positions 3 and 13.

A further aspect of the invention is directed to the cyclic peptide comprising an amino acid sequence having at least 85% identity with the amino acid sequence CAEFRHDSGYEVCH (SEQ ID NO: 14) or variant thereof wherein the peptide comprises the cysteine residues at positions 1 and 13 and the phenylalanine residue at position 4, wherein the peptide is cyclized via the cysteine residues at positions 1 and 13.

In a further aspect of the invention the cyclic peptide comprises the amino acid sequence having at least 85% identity with the amino acid sequence DACFRHDSGYECHH (SEQ ID NO: 4) or variant thereof wherein the peptide comprises the cysteine residues at positions 3 and 12 and the phenylalanine residue at position 4, wherein the peptide is cyclized via the cysteine residues at positions 3 and 12.

In a further aspect of the invention the cyclic peptide comprises the amino acid sequence having at least 85% identity with the amino acid sequence DACFRHDSGYEVCH (SEQ ID NO: 13) or variant thereof wherein the peptide comprises the cysteine residues at positions 3 and 13 and the phenylalanine residue at position 4, wherein the peptide is cyclized via the cysteine residues at positions 3 and 13.

In an additional aspect of the invention the cyclic peptide comprises the amino acid sequence DAEFRHDSGYEVHH (SEQ ID NO: 3) or variant thereof, wherein one of the amino acid residues at position 1, 2, 3 or 5 is substituted with a cysteine residue and wherein one of the amino acid residues at position 10, 11, 12 or 13 is substituted with a cysteine residue, such that the peptide comprises two cysteine residues and the peptide is cyclized between the two cysteine residues.

Preferably in one embodiment the cyclic peptide comprises the amino acid sequence DAEFRHDSGYEVHH (SEQ ID NO: 3) or variant thereof, wherein the amino acid residue at position 1 is substituted with a cysteine residue and wherein one of the amino acid residues at position 10, 11, 12 or 13 is substituted with a cysteine. Preferably the amino acid resides at position 13 is substituted with a cysteine residue.

Alternatively, in one embodiment the cyclic peptide comprises the amino acid sequence DAEFRHDSGYEVHH (SEQ ID NO: 3) or variant thereof, wherein one of the amino acid residues at position 2, 3 or 5 is substituted with a cysteine residue and wherein one of the amino acid residues at position 10, 11, 12 or 13 is substituted with a cysteine. Preferably there is at least 7 amino acid between the two cysteine residues present in the sequence, more preferably there is between 7 and 10, even more preferably there is 8 or 9 amino acid residues between the two cysteine residues present in the sequence. In one embodiment the peptide does not comprise cysteine residues at both positions 5 and 12 or at positions 3 and 10.

A further aspect of the invention relates to a pharmaceutical composition comprising a cyclic peptide described above and a pharmaceutically acceptable carrier. Preferably the composition further comprises an adjuvant. The composition may be an immunogenic composition. In one embodiment those compositions may be a vaccine composition.

Aspects of the invention also relate to the cyclic peptide for use as a medicament. One embodiment relates to a method of treating a neurodegenerative disease comprising administering a cyclic peptide or composition as described above to an individual in need thereof. Preferably the neurodegenerative disease is Alzheimer's disease.

A further embodiment of the invention relates to a method for inducing an immune response in a subject comprising administering a cyclic peptide or composition as described above to the subject, i.e. a cyclic peptide that adopts the hairpin structure of amyloid-beta or composition comprising the same. Preferably an immune response that generates antibodies against amyloid-beta, more preferably the amyloid beta is in the form of low molecular weight amyloid-beta oligomers, and the method is for inducing an immune response against low molecular weight amyloid-beta oligomers.

One embodiment of the invention relates to a cyclic peptide as described above for use in treating a neurodegenerative disease. Preferably the neurodegenerative disease is Alzheimer's disease.

A further embodiment of the invention relates to a cyclic peptide as described above for use in inducing an immune response in a subject. Preferably an immune response that generates antibodies against amyloid-beta, more preferably the amyloid-beta is in the form of low molecular weight amyloid-beta oligomers, and the use is for inducing an immune response against low molecular weight amyloid-beat oligomers.

A further embodiment of the invention relates to a cyclic peptide for the manufacture of a medicament for treating a neurodegenerative disease, such as Alzheimer's disease and/or for inducing an immune response, preferably to produce antibodies against amyloid-beta oligomers, preferably low molecular weight amyloid-beta oligomers, i.e. a cyclic peptide that adopts the hairpin structure of amyloid-beta.

A further aspect of the invention relates to a method of producing a cyclic peptide as described above comprising the steps of:
(a) synthesizing a linear peptide comprising the sequence of the peptide; and
(b) cyclizing the linear peptide via the cysteine residues to obtain the cyclic peptide according to formula (I).

A further aspect of the invention relates a method for the generating an antibody that recognizes low molecular weight oligomers of amyloid beta comprising:
(a) Immunizing an animal with a cyclic peptide or variant thereof as described above; and
(b) obtaining the antibodies generated by the immunization in step (a).

The method can further comprise step (c) comprising screening the antibodies obtained in step (b) for their recognition of low molecular weight oligomers of amyloid beta. Preferably the antibodies are also screened for their ability to not bind, or not bind significantly with, Aβ1-42, Aβ1-40 and/or Aβ1-38. Preferably the antibodies are screened for their ability to specifically recognise low molecular weight oligomers of amyloid beta, preferably low molecular weight AβpE3-x and Aβ4-x, more preferably AβpE3-42 and Aβ4-42. Preferably the method comprises immunising an animal with a cyclic peptide having the sequence of SEQ ID NO: 4, 13 or 14.

A further aspect of the invention comprises an antibody obtainable by the above method. The antibody obtained may be used in a composition, such as vaccine composition. The antibody could be used for the treatment of Alzheimer's disease.

Other aspects and embodiments of the invention are described in more detail below.

### BRIEF DESCRIPTION OF THE FIGURES

FIG. 1 shows the structure of TAP01 Fab;

FIG. 2 shows the structure of TAP01-pE3-14 Fab;

FIG. 3 shows the (a) the structure of pGlu3-14 and (b) the TAP01-pGlu3-14 amyloid peptide structure;

FIG. 4 shows the comparison of structures of TAP01-pE3-14 and TAP01_01-pE3-14;

FIG. 5 shows the structure of TAP01-1-14 cyclised peptide;

FIG. 6 shows the comparison of structures of (A) 1-14 (cysteines 3, 12) and (B) pGlu3-14 cyclic amyloid peptides;

FIG. 7 shows the binding ELISA data for binding of comparator antibodies (Bapineuzumab, solanezumab, BAN2401, ProBioDrug 6_1_6, ProBioDrug 24_2_3) and Tap01 to disulphide bridged 1-14 cyclic peptide 3, 12;

FIG. 8 shows the binding ELISA data for binding of animal sera to disulphide bridged 1-14 cyclic peptide 3, 12;

FIG. 9 shows the binding ELISA data for binding of animal sera to Aβ1-42 peptide;

FIG. 10 shows the binding ELISA data for binding of animal sera to AβpE3-42 peptide;

FIG. 11 shows the binding ELISA data for binding of animal sera to Aβ4-42 peptide;

FIG. 12 shows the binding ELISA data for binding of animal sera to KLH antigen;

FIG. 13 shows the binding ELISA data for binding of animal sera to thioacetal bridged 1-14 cyclic peptide 3,12;

FIG. 14 shows the binding ELISA data for binding of animal sera to Aβ1-42 peptide;

FIG. 15 shows the binding ELISA data for binding of animal sera to AβpE3-42 peptide;

FIG. 16 shows the binding ELISA data for binding of animal sera to Aβ4-42 peptide;

FIG. 17 shows the immunostaining of AD mouse model brain sections with M2 anti-serum. SXFAD mostly Aβ 1-42 and plaques; Tg4-42 only Aβ 4-42 and TBA42 only pyroglutamate Aβ 3-42;

FIG. 18 shows the immunostaining of AD mouse model brain sections with M4 anti-serum. SXFAD mostly Aβ 1-42 and plaques; Tg4-42 only Aβ 4-42 and TBA42 only pyroglutamate Aβ 3-42;

FIG. 19 shows the effects of TAP01_04 (cloned as MoG1K) on 18F-FDG uptake in young and aged Tg4-42 mice;

FIG. 20 shows the binding ELISA data for binding of (a) TAP01 (MoG1K) antibody and (b) MRCT-Control IgG1 antibody (cloned as MoG1K) to thioacetal bridged cyclic peptide variants;

FIG. 21 shows the binding ELISA data for binding of (a) TAP01 (MoG1K) antibody and (b) MRCT-Control IgG1 antibody (cloned as MoG1K), to thioacetal bridged cyclic peptide variants;

FIG. 22 shows the binding ELISA data for binding of comparator antibodies (Bapineuzumab, solanezumab, BAN2401, ProBioDrug 6_1_6, ProBioDrug 24_2_3) and TAP01 HuG4K to thioacetal bridged cyclic peptide variants;

FIG. 23 shows the proline mutated peptide binding to TAP01 antibody using Biacore T200;

FIG. 24 shows reduced cortical plaque load in immunized 5XFAD mice after passive immunization with TAP01 antibodies. Plaque load analysis of TAP01_4 (MoG1K) immunized 5XFAD mice compared to IgG1 injected 5XFAD mice. (a) Immunostaining with an antibody against pan-Aβ showing significant reduced plaque load in TAP01_04 immunized mice as compared to IgG control, TAP01_01 and TAP01_02 treated mice; (b) Immunostaining with an antibody against pyroglutamate Aβ3-x showing significant reduced plaque load in TAP01_04 immunized mice as compared to IgG control. No significant difference was observed to mice immunized with TAP01_01 and TAP01_02; (c) Staining with Thioflavin S (d) shows immunostaining with TAP01 (NT4X) showing significant reduced plaque load in TAP01_04 immunized mice as compared to IgG control. No significant difference was observed to mice immunized with TAP01_01 and TAP01_02.

FIG. 25 shows the binding ELISA data for binding of (a) TAP01 (MoG1K) antibody (b) MRCT-Control IgG1 antibody (cloned as MoG1K), to thioacetal bridged cyclic peptide variants;

FIG. 26 shows the binding ELISA data for binding of (a) Bapineuzumab to thioacetal bridged cyclic peptide variants in comparison with (b) MRCT-Control IgG1 antibody (cloned as HuG1K);

FIG. 27 shows in vivo amyloid-plaque imaging with tracer fluorbetaben of transverse sections of mouse brain. (A) Untreated wildtype control mouse brain with no fluorbetaben retention signal; (B) Untreated 5XFAD with strong fluorbetaben retention signal pointing to a high amyloid-plaque load in brain and (C) 5XFAD mice after active immunization show no fluorbetaben retention signal pointing to a significantly reduced amyloid-plaque load in brain;

FIG. 28 shows statistical analysis of the fluorbetaben retention signal as a marker for amyloid-plaque load in mouse brain. Statistical evaluation with ANOVA (p<0.0001, F=21.39) and Bonferroni corrected comparison between groups: wildtype (WT) cortex versus 5XFAD cortex (p<0.001), WT hippocampus versus 5XFAD hippocampus (p<0.001), WT amygdala versus 5XFAD amygdala (p<0.001). WT cortex versus 5XFAD cortex treated (not significant), WT hippocampus versus 5XFAD hippocampus treated (not significant), WT amygdala versus 5XFAD amygdala treated (not significant). 5XFAD cortex versus 5XFAD cortex treated (p<0.01), 5XFAD hippocampus versus 5XFAD hippocampus treated (p<0.001), 5XFAD amygdala versus 5XFAD amygdala treated (p<0.001). 5XFAD treated=immunized 5XFAD with cyclic peptide.

FIG. 29 shows immunostaining and quantitative assessment of plaque load in 5XFAD mouse cortical brain comparing passive immunization with TAP01_04 (MoG1K) and active immunization with the cyclic peptide. Exemplary staining with pan-Aβ antibodies (A) is shown for 5XFAD mice following treatment with IgG1, passive immunisation with TAP01_04 and active immunisation with cyclised Aβ peptides. Quantification of plaque load (B) was assessed using antibodies against pan-Abeta, pyroglutamate Aβ3-X, Thioflavin S and N-truncated Aβ, and demonstrated a strong reduction of plaques in 5XFAD mice treated by active immunization. Mice treated by TAP01_04 and active immunization showed similar reducing effects on plaques stained with all Abeta-antibodies and Thioflavin S. ANOVA with Bonferroni's multiple comparison test for plaque staining against pan-Abeta (F=65.20, p<0.0001, R squared 0.6287), pyroglutamate Aβ3-X (F=23.32, p<0.0001, R squared 0.3570), Thioflavin S (F=17.17, p<0.0001, R squared 0.3291) and N-truncated Aβ (F=89.17, p<0.0001, R squared 0.6316) is shown (mean+SEM).

FIG. 30 shows the effect of active immunisation with cyclised Aβ peptides in vivo brain glucose metabolism in 5XFAD mice. (A) Exemplary coronal, transverse and sagittal views of glucose uptake by 18F-FDG-PET/MRI imaging. (B) Quantitative analysis. Glucose uptake was assessed by 18F-FDG-PET/MRI imaging in active immunized 5XFAD (n=5), two 5XFAD mouse control and two wildtype mice (all female, age 4.5-5.5 months of age). Quantitative analysis of the signal intensities demonstrated that immunized 5XFAD mice showed a significant rescue of the brain glucose metabolism in most brain areas analyzed demonstrating a therapeutic effect in synaptic and neuronal activities. ANOVA comparison test (F=10.37, p<0.0001, R squared=0.7352). Significant differences using t-test are shown (mean+SEM). A, Amygdala; Bs, Brain Stem; C, Cortex; Cb, Cerebellum; H, Hypothalamus; Hc, Hippocampus; Hg, Harderian gland; M, Midbrain; O, Olfactory Bulb; S, Septum/Basal Forebrain; St, Striatum; T, Thalamus.

FIG. 31 shows the effect of active immunisation with cyclised Aβ peptides in comparison with passive immunization with TAP01_04 (MoG1K) in Tg4-42 mice. (A) Hippocampus-dependent learning and memory loss in aged Tg4-42 is demonstrated by the probe trial of the Morris Water Maze test. Both passive immunization with TAP01_04 and active immunization with the cylised Aβ peptide rescued memory deficits in Tg4-42 mice. ANOVA with Bonferroni's multiple comparison test (F=13.27, p<0.0001, R squared=0.646). T-test with mean+SEM are shown. (B) Aged Tg4-42 mice develop a significant neuron loss in the CA1 layer of the hippocampus. The mean number (+SEM) of neurons on IgG1 treated mice is 128687+13035, whereas the mean number (+SEM) of neurons in TAP01_04 treated mice is significantly higher with 194310+22572 and the mean number (+SEM) of neurons in active immunized mice is significantly higher as well with 185858+39180. ANOVA with shown Bonferroni's multiple comparison test (F=8.125, p<0.001, R squared=0.5556).*=p<0.05; **p<0.01; ***=p<0.001.

FIG. 32 shows binding ELISA data for binding of 5XFAD mouse sera to cyclised Aβ peptide.

FIG. 33 shows binding ELISA data for binding of Tg4-42 mouse sera to cyclised Aβ peptide.

FIG. 34 shows binding ELISA data for binding of control antibodies HuMRCT MoG1K and MoMRCT HUG1K, comparator antibody Bapineuzumab and TAP01 MoG1K to cyclic peptide 3, 13.

### DETAILED DESCRIPTION

The present invention relates to a non-naturally occurring peptide, i.e. synthetic peptide, that mimics a conformational epitope naturally found in the pE3-X amyloid peptides and which specifically binds to antibodies that bind the epitope of the pE3-X amyloid peptides. The cyclic peptide may be used for active immunisation of a subject for the generation of antibodies specific to amyloid-beta, in particular to low molecular weight oligomers of amyloid-beta. A hairpin structure is found at the N-terminal region of the pE3-X amyloid peptides. This region has been found to be the epitope for antibodies that bind low molecular weight oligomers of amyloid-beta.

The cyclic peptides according to the invention are based on amino acid resides 1-14 of amyloid-beta protein with two of the residues found in this sequence replaced with cysteine residues via which the peptide is cyclised. The cyclic peptides of the invention mimic the hairpin structure found in amyloid beta.

The cyclic peptides mimic the hairpin structure identified in pE3-X amyloid-beta and which has been identified as the binding site for antibodies such as the mouse TAP01 antibody (also known as NT4X) and the humanised TAP01_01, TAP01_02, TAP01_03, and TAP01_04 antibodies (also known as NT4X_SA, NT4X_S7A, NT4X_S71A and NT4X_S71H respectively and as described in WO2011/151076 and WO2020/070225). These anti-amyloid-beta antibodies have been shown to specifically bind N-terminal truncated amyloid peptide (AβpE3-x or Aβ4-x). These antibodies do not display significant binding to full length amyloid peptides or amyloid Aβ1-42.

The cyclic peptide as described herein are variant peptides based on amino acids 1-14 of amyloid-beta DAEFRHDSGYEVHH (SEQ ID NO: 3), wherein two amino acids of the naturally occurring sequences are replaced with cysteine residues via which the peptide is cyclised. Preferably one of the cysteine residues replaces the amino acid at position 1, 2, 3, or 5 and the other cysteine replaces an amino acid at residue at position 10, 11, 12 or 13.

In one embodiment a cyclic peptide described herein comprises an amino acid sequence having the sequence of formula (I) (SEQ ID NO: 1) or variant thereof:

X₁X₂X₃FX₄HDSGX₅X₆X₇X₈H (I)
wherein:
   X₁ is absence or any amino acid; and
   X₂ is alanine or cysteine;
   X₃ is glutamic acid or cysteine;
   X₄ is arginine or cysteine;
   X₅ is tyrosine or cysteine;
   X₆ is glutamic acid or cysteine;
   X₇ is valine or cysteine; and
   X₈ is histidine or cysteine
      wherein only one of X₁, X₂, X₃ and X₄ is cysteine and wherein only one of X₅, X₆, X₇ and X₈ is cysteine and the peptide is a cyclized via the cysteine residue at position 1, 2, 3, or 5 and the cysteine residue at position 10, 11, 12 or 13. The cyclic peptide may comprise only two cysteine residues, via which peptide is cyclised.

Preferably there is at least 7 amino acid between the two cysteine residues present in the sequence, more preferably there is from 7 to 11 amino acid residues, even more preferably there is 8 to 11 amino acid residues between the two cysteine residues present in the cyclic peptide.

The cyclic peptide is preferably not cyclised via both the terminal amino acids of the peptide sequences. Preferably X₁ is present. Preferably the cyclic peptide is not cyclised via the C-terminal amino acid. In one embodiment X₁ is cysteine, and preferably the peptide is cyclised via the cysteine at position 1 and a cysteine at position 13. In further embodiment preferably X₁ is proline or aspartic acid, preferably aspartic acid and the peptide is not cyclised via either of the terminal amino acids of the peptide residues. Cyclising the peptide via at least one internal cysteine resides, in particular, at position 1, 2, 3, or 5 and position 10, 11, 12 or 13 helps provide a stable peptide that can mimic the hairpin structure found in pE3-X amyloid-beta.

In one embodiment the peptide is cyclised via a cysteine residue at position 1, i.e. X₁ is cysteine and X₂ is alanine, and a cysteine at position 10, 11, 12 or 13, preferably at position 13. Preferably the cyclic amino acid comprises a sequence wherein X₁ is cysteine, X₂ is alanine, X₃ is glutamic acid, X₄ is arginine, X₅ is tyrosine, X₆ is glutamic acid, X₇ is valine and X₈ is cysteine wherein the peptide is a cyclized via the two cysteine residues. For example, for peptide is cyclised via the cysteines at X₁ and X₈. For example, the cyclic peptide can comprise or consists of the amino acid sequence CAEFRHDSGYEVCH (SEQ ID NO: 14) wherein the peptide is cyclised via the cysteine residues at positions 1 and 13.

Alternatively, in one embodiment preferably the cyclic amino acid comprises a sequence wherein X₁ is absence or any amino acid; and
a) X₂ is alanine, X₃ is cysteine, X₄ is arginine, X₅ is tyrosine, X₆ is glutamic acid, X₇ is cysteine and X₈ is histidine;
b) X₂ is cysteine, X₃ is glutamic acid, X₄ is arginine, X₅ is tyrosine, X₆ is glutamic acid, X₇ is cysteine and X₈ is histidine;
c) X₂ is cysteine, X₃ is glutamic acid, X₄ is arginine, X₅ is cysteine, X₆ is glutamic acid, X₇ is valine and X₈ is histidine;
d) X₂ is cysteine, X₃ is glutamic acid, X₄ is arginine, X₅ is tyrosine, X₆ is glutamic acid, X₇ is valine and X₈ is cysteine
e) X₂ is cysteine, X₃ is glutamic acid, X₄ is arginine, X₅ is tyrosine, X₆ is cysteine, X₇ is valine and X₈ is histidine;
f) X₂ is alanine, X₃ is cysteine, X₄ is arginine, X₅ is tyrosine, X₆ is cysteine, X₇ is valine and X₈ is histidine;
g) X₂ is alanine, X₃ is glutamic acid, X₄ is cysteine, X₅ is tyrosine, X₆ is glutamic acid, X₇ is cysteine and X₈ is histidine;
h) X₂ is alanine, X₃ is cysteine, X₄ is arginine, X₅ is cysteine, X₆ is glutamic acid, X₇ is valine and X₈ is histidine; or
i) X₂ is alanine, X₃ is cysteine, X₄ is arginine, X₅ is tyrosine, X₆ is glutamic acid, X₇ is valine and X₈ is cysteine;
   wherein the peptide is a cyclized via the two cysteine residues. For example for peptide (a) the sequence is cyclised via the cysteines at X₃ and X₇, for peptide (b) the sequence is cyclised via the cysteines at X₂ and X₇, for peptide (c) the sequence is cyclised via the cysteine at X₂ and X₅, for peptide (d) the sequence is cyclised via the cysteines at X₂ and X₈, for peptide (e) the sequence is cyclised via the cysteines at X₂ and X₆, for peptide (f) the sequence is cyclised via the cysteines at X₃ and X₆, for peptide (g) the sequence is cyclised via the cysteines at X₄ and X₇; for peptide h) the sequence is cyclised via cysteine at X₃ and X₅, and for peptide i) the sequence is cyclised via cysteine at X₃ and X₈. Preferably in one embodiment X₁ is present and is selected from proline or aspartic acid. More preferably X₁ is aspartic acid,

For example, the cyclic peptide can comprise or consists of the amino acid sequence:

(SEQ ID NO: 4)
a) DACFRHDSGYECHH;
(SEQ ID NO: 5)
b) DCEFRHDSGYECHH;
(SEQ ID NO: 10)
c) DCEFRHDSGCEVHH;
(SEQ ID NO: 12)
d) DCEFRHDSGYEVCH;
(SEQ ID NO: 9)
e) DCEFRHDSGYCVHH;
(SEQ ID NO: 8)
f) DACFRHDSGYCVHH;
(SEQ ID NO: 7)
g) DAEFCHDSGYECHH;
(SEQ ID NO: 11)
h) DACFRHDSGCEVHH;
or
(SEQ ID NO: 13)
i) DACFRHDSGYEVCH.

The peptide is cyclised via the two cysteine residues located at positions 2, 3 or 5 and 10, 11, 12, or 13. Preferably the cyclic peptide comprises the sequence DACFRHDSGYECHH (SEQ ID NO: 4) wherein the peptide is cyclised via the cysteine residues at positions 3 and 12 or DACFRHDSGYEVCH (SEQ ID NO: 13) wherein the peptide is cyclised via the cysteine residues at positions 3 and 13.

The present invention also relates to cyclic peptides comprising the sequence of formula (I) as described above, wherein the cyclic peptide does not comprise cysteine residues at both positions 5 and 12 or at both positions 3 and 10. In particular, the cyclic peptide does not comprise or consist of a peptide have the sequence of SEQ ID NO: 7 or SEQ ID NO: 11.

Variant cyclic peptides are also provided. Variant cyclic peptides have the same or similar function as their reference peptide, i.e. are functionally equivalent cyclic peptides, and adopt the hairpin structure of amyloid-beta. A cyclic peptide as described herein that is a variant of a reference sequence, such as a reference sequence described above, may have 1 or more amino acid residues altered relative to the reference sequence. For example, 3 or fewer amino acid residues may be altered relative to the reference sequence, preferably 2 or fewer, or 1 amino acid residue may be altered relative to the reference sequence. An amino acid residue in the reference sequence may be altered or mutated by insertion, deletion or substitution, preferably substitution for a different amino acid residue. Preferably the substitutions are conservative amino acid substitutions. Conservative amino acid sequence modifications are modification which do not affect or alter the characteristics of the cyclic peptide, for example maintain the confirmation of cyclic peptide, and preferably maintain the immunogenicity of the peptide, preferably maintain the ability to induce an immune response which may generate anti-amyloid-beta antibodies, preferably those which specifically bind low molecular weight AB oligomers.

Conservative amino acid substitutions include ones in which the amino acid residue is replaced with an amino acid residue having a similar side chain. Families of amino acid residues having similar side chains have been defined in the art. Substitution include substitutions with any of the twenty naturally occurring (or ‘standard’) amino acids or variants thereof, such as e.g. D-amino acids, or any variants that are not naturally found in proteins. Non-natural amino acids have been defined in the art.

A cyclic peptide as described herein that is a variant of a reference sequence may share at least 85% sequence identity with the reference sequence, at least 90%, at least 95% at least 98% or at least 99% sequence identity with the reference sequence, e.g. SEQ ID NO: 4, 5, 7, 9, 10, 11, 12, 13 or 14. A cyclic peptide as described herein that is a variant of a reference sequence maintains at least one internal cysteine residue, i.e. comprises at least one non-terminal cysteine residues via which the peptide is cyclised. The cysteine residues may be positioned one at the N-terminal region of the peptide and the other at the C-terminal region of the peptide, wherein the cysteines residues are not both positioned at the terminus of the sequence, i.e. the cyclic peptide comprises at least one free N- and C-terminal residues such that the peptide is not cyclised in a head-to-tail form. In one embodiment the cysteine residue is not present as the C-terminal residue of the sequence. A cyclic peptide as described herein that is a variant of a reference sequence, i.e. SEQ ID NO: 4, 5, 7, 9, 10, 11, 12, 13 or 14 maintains two cysteine residues, one which is not present at the terminus of the sequence, i.e. comprises at least one non-terminal cysteine residues via which the peptide is cyclised. In one embodiment neither cysteine residues are positioned at the terminus of the sequence. A cyclic peptide as described herein that is a variant of a reference sequence, i.e. SEQ ID NO: 4, 5, 7, 9, 10, 11, 12 or 13 maintains the two internal cysteine residues, i.e. comprises at two non-terminal cysteine residues via which the peptide is cyclised,

Preferably cysteine residues are maintained at positions 1, 2, 3 or 5 and 10, 11, 12 or 13, preferably at positions 1 and 13, positions 3 and 12, or at positions 3 and 13. Preferably the phenylalanine residue at position 4 of the reference sequence is also maintained. For example the cyclic peptide described herein may comprise an amino acid sequence having at least 85% sequence identity, at least 90%, at least 95%, at least 98%, at least 99% sequence identity with the sequence CAEFRHDSGYEVCH (SEQ ID NO: 14), the sequence DACFRHDSGYECHH (SEQ ID NO: 4), or the sequence DACFRHDSGYEVCH (SEQ ID NO: 13) wherein the peptide comprises the cysteine residues at positions 1 and 13, positions 3 and 12 or at positions 3 and 13 and the phenylalanine residue at position 4 and wherein the peptide is cyclized via the cysteine residues at positions 1 and 13, positions 3 and 12 or at positions 3 and 13. In such a variant cyclic peptide the cysteine residues at positions 1 and 13, positions 3 and 12 or at positions 3 and 13 and the phenylalanine residue at position 4 are maintained, whilst conservative amino acid substitutions are introduced into the sequence at other positions. In one embodiment the cysteine residues at positions 1 and 13 are maintained in the variant sequence. In another embodiment the cysteine residue at position 3 and the cysteine residues at positions 12 or 13 are maintained in the variant sequence.

Sequence identity is commonly defined with reference to the algorithm GAP (Wisconsin GCG package, Accelerys Inc, San Diego USA). GAP uses the Needleman and Wunsch algorithm to align two complete sequences that maximizes the number of matches and minimizes the number of gaps. Generally, default parameters are used, with a gap creation penalty=12 and gap extension penalty=4. Use of GAP may be preferred but other algorithms may be used, e.g. BLAST (which uses the method of Altschul et al. (1990) J. Mol. Biol. 215: 405-410), FASTA (which uses the method of Pearson and Lipman (1988) PNAS USA 85: 2444-2448), or the Smith-Waterman algorithm (Smith and Waterman (1981) J. Mol Biol. 147: 195-197), or the TBLASTN program, of Altschul et al. (1990) supra, generally employing default parameters. In particular, the psi-Blast algorithm may be used (Nucl. Acids Res. (1997) 25 3389-3402). Sequence identity and similarity may also be determined using Genomequest™ software (Gene-IT, Worcester Mass. USA). Sequence comparisons are preferably made over the full-length of the relevant sequence described herein.

As used throughout the present application, the amino acid positions with respect to the cyclic peptide are given in reference to the sequence of a peptide having the sequence of DAEFRHDSGYEVHH (SEQ ID NO: 3). Therefore the wording “the amino acid at position “x”” of the cyclic peptide or similar thus means the amino acid corresponding to the amino acid at position “x” in the preferred cyclic peptide having SEQ ID NO: 3. The amino positions with respect to the full length amyloid-beta peptides, and variants thereof including N-truncated variants, e.g. 1-40, p3-42, and 4-42 are given in reference to the sequence of the full length peptide having the sequence of Aβ 1-42 (SEQ ID NO: 18). Therefore, the wording “the amino acid at position “x”” of the N-terminal truncated p3-42 peptide or similar thus means the amino acid corresponding to the amino acid at position “x” in the preferred full length peptide having SEQ ID NO: 18. Note that, the numbering system used throughout this application starts from the N-terminal amino acid.

The cyclic peptides described herein can comprise, consist essentially of or consist of the variant amino acid sequences of the peptides or variants thereof described here. In a preferred embodiment the cyclic peptide comprises not more than 16 amino acids, preferably not more than 15 amino acids. More preferably the peptide comprises not more than 14 amino acids. In a preferred embodiment the cyclic peptide consists or consists essentially of the amino acid sequences described herein, i.e. the cyclic peptide consists or consists essentially of the sequence shown in formula (I), formula (II) or SEQ ID NOs: 4, 5, 7, 8, 9, 10, 11, 12, 13 or 14, or a variant thereof.

By cyclization or “is cyclised” or similar it is meant the peptide is or is made into a cyclic form. The term “cyclic” means that at least some of the constituent residue of the peptide form a ring. The cyclic peptides of the invention are cyclised via at least one internal amino acid, i.e. are not cyclised in a head-to-tail form. Preferably the peptides of the invention are cyclised via internal amino acids.

Cyclisation of the peptide via the cysteine residues constrains the peptide into a structure that mimics the hairpin structure identified in pE3-X amyloid-beta.

Cyclization of the peptide is obtained via formation of a bridge through incorporation of the two cysteine residues into the sequence. The cysteine residues replace the corresponding amino acid in the naturally occurring sequence. Preferably cyclization can be formed by side-chain to side-chain cyclization. Peptides of the invention may be cyclised directly or indirectly via the thiol side-chains of the cysteine residues. For example, side-chain to side-chain cyclization can be obtained via formation of a bridge of the formula —S—(—CH₂-)n-S—, wherein n=0, 1 or 2. Preferably the bridge has the formula —S—S— or —S—CH₂—S—, wherein S are the thiol residues of the connected cysteine residues. More preferably the bridge has the formula —S—CH₂—S—, preferably between cysteine residues located at positions 1 and 13 of the peptide, between cysteine residues located at positions 3 and 12 of the peptide or between cysteine residues located at positions 3 and 13 of the peptide. Suitable methods for cyclising peptides via cysteine residues are known in the art e.g. using thiol oxidation, optionally with the introduction with a methylene bridge. See also for example [Kourra C and Cramer N, Chem. Sci., 2016, 7, 7007-7012]. Other bridges such as a thioether bridge (—CH₂—S—) are also encompassed by the invention.

The cyclic peptide shows binding specificity to the TAP01 and TAP01_01 antibodies (as described in WO2013/167681 and WO2020/070225). The cyclic peptide mimics the N-terminal epitope found on the p3-42 amyloid-beta, having a hairpin structure, which these antibodies bind.

In a preferred embodiment, the cyclic peptide specifically binds antibody molecules that specifically recognizes soluble low molecular weight AβpE3-X oligomers, i.e. do not bind antibodies that bind specifically to high molecular weight oligomers of Aβ pE3 peptides. As used herein, the term “low molecular weight oligomers” refers to soluble oligomers made up of 3 to 6 AβpE3-X, preferably trimeric and tetrameric Aβp3-x or Aβ4-x oligomers, wherein X is 38, 40, 42. Preferably low molecular weight oligomers of Aβp3-x or Aβ4-x are at least trimeric oligomeric and have a size of less than 15 kDA.

The antibodies the cyclic peptide may specifically bind to N-terminal truncated amyloid peptides, for example pyroglutamate (pE) modified amyloid peptides (also referred to as AβpE3-x, AβpGlu3-x, 25 Aβ(Glp3)₃-x, and p3-x), such as AβpE3-38, AβpE3-40, AβpE3-14 and AβpE3-42, and non-pyroglutamate modified amyloid peptides, such as Aβ4-38, Aβ4-40, Aβ4-14 and Aβ4-40. The antibodies such as TAP01 and TAP01_01 may display no specific binding to full-length amyloid peptides or amyloid peptides without N terminal truncations (Aβ1-x), such as Aβ1-42, Aβ1-38, Aβ1-40 or Aβ1-14.

In a preferred embodiment, antibodies, which the cyclic peptide described herein bind, may specifically bind to the amyloid peptides AβpE3-42 and Aβ4-42. The antibody may show no specific binding or substantially no specific binding to monomers and dimers of A131-42.

Specific binding or “specifically recognising” refers to the situation in which an antibody will not show any significant binding to molecules other than its specific epitope on an antigen.

For example, the term “specifically recognising” or the like, as used herein, is intended to mean that the binding molecule, i.e. antibody, specifically binds and/or detects (i.e. recognises) soluble low molecular weight oligomers of N-terminal truncated amyloid peptides, i.e. Aβp3-x or Aβ4-x, wherein X is 42, 40 or 38 e.g. Aβp3-42 or Aβ4-42. The antibodies do not recognise or bind monomers or dimers of Aβ1-40 or high molecular weight oligomers. Accordingly, the antibodies preferably recognise the conformational epitope formed in trimeric or tetrameric Aβp3-42 oligomers. Antibodies that specifically recognise low molecular weight oligomers of amyloid-beta and display no or little binding to full-length amyloid peptide, include but are not limited to TAP01 and TAP01_01.

The affinity of an antibody described herein is the extent or strength of binding of antibody to epitope or antigen, including its binding to the cyclic peptide defined herein. The dissociation constant, Kd, and the affinity constant, Ka, are quantitative measures of affinity. Kd is the ratio of the antibody dissociation rate (k_{off}), how quickly it dissociates from its antigen, to the antibody association rate (kₒₙ) of the antibody, how quickly it binds to its antigen. The binding of an antibody to its antigen is a reversible process, and the rate of the binding reaction is proportional to the concentrations of the reactants. At equilibrium, the rate of [antibody][antigen] complex formation is equal to the rate of dissociation into its components [antibody]+[antigen]. The measurement of the reaction rate constants may be used to define an equilibrium or affinity constant, Ka (Ka=1/Kd). The smaller the Kd value, the greater the affinity of the antibody for its target. Most antibodies have Kd values in the low micromolar (10⁻⁶) to nanomolar (10⁻⁷ to 10⁻⁹) range. High affinity antibodies are generally considered to be in the low nanomolar range (10⁻⁹) with very high affinity antibodies being in the picomolar (10⁻¹²) range.

In some embodiments, an anti-Aβ antibody (which the cyclic peptide binds), binds (e.g. specifically binds) to amyloid peptides AβpE3-42 and Aβ4-42 with an affinity constant or Ka of at least 2×10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, or at least 10⁷ M⁻¹.

In some embodiments, an anti-Aβ antibody (which the cyclic peptide binds), may have a dissociation constant or Kd from amyloid peptides AβpE3-42 and Aβ4-42 of less than 5×10² M, less than 10-²M, less than 5×10⁻³ M, less than 10⁻³ M, less than 5×10⁻⁴ M, less than 10⁻⁴ M, less than 5×10⁻⁵ M⁻¹, less than 5×10⁻⁵, less than 5×10⁻⁶, less than 10⁻⁶, or less than 5×10⁻⁷ M.

Specific binding of an antibody means that the antibody exhibits appreciable affinity for a particular antigen or epitope and, generally, does not exhibit significant cross-reactivity. An antibody that “does not exhibit significant cross-reactivity” is one that will not appreciably bind to an undesirable entity (e.g., an undesirable proteinaceous entity). An antibody specific for a particular epitope will, for example, not significantly cross-react with remote epitopes on the same protein or peptide. Specific binding i.e., k_{off}, kₒₙ, Ka and Kd, of an antibody described herein may be determined according to any art-recognized means for determining such binding.

Binding of the anti-Aβ antibody may be determined using standard techniques, such as an ELISA or Surface Plasmon Resonance. Suitable ELISA techniques are well known in the art. For example, immobilised amyloid peptide may be contacted with the antibody in an IgG1 format and washed one or more times in 0.1% non-ionic detergent, such as polysorbate 20 (Tween 20), to remove unbound antibody. Antibody bound to the immobilised peptide may then be detected using any convenient technique, for example using a secondary antibody bound to a detectable label, such as HRP.

The invention further provides cyclic peptides described herein linked to a carrier, preferably a carrier protein. Preferably the peptide is linked to a carrier by chemical crosslinking. The cyclic peptide may be conjugated to a carrier protein, including but not limited to keyhole limpet hemocyanin (KLH), serum albumin (such as bovine serum albumin, BSA) or ovalbumin, an immunoglobin FC domain, tetanus toxoid, diphtheria toxoid or combinations thereof. The carrier peptide may be connected directly to the cyclic peptide or via a linker. The peptide may be linked to the carrier protein via standard techniques in the art.

A cyclic peptide as described herein may be useful in therapy. For example, the cyclic peptide protein may be administered to an individual for the treatment of a neurological disease. The cyclic peptide will usually be administered in the form of a pharmaceutical composition, which may comprise at least one additional component in addition to the cyclic peptide.

Cyclic peptides and compositions described herein may be administered for therapeutic and/or prophylactic treatment by parenteral, topical, intravenous, oral, gastric, subcutaneous, intra-arterial, intracranial, intraperitoneal, intranasal or intramuscular methods, as described herein. Intramuscular injection or intravenous infusion are preferred for administration of cyclic peptides.

The pharmaceutical compositions may comprise, in addition to the cyclic peptide described herein, a pharmaceutically acceptable excipient, carrier, buffer, stabilizer and/or other materials well known to those skilled in the art. The term “pharmaceutically acceptable” as used herein pertains to compounds, materials, compositions, and/or dosage forms which are, within the scope of sound medical judgement, suitable for administering to the subject (e.g., human) and will cause any unwanted or harmful effects to the subject. Each carrier, excipient, etc. must also be “acceptable” in the sense of being compatible with the other ingredients of the formulation. The precise nature of the carrier or other material will depend on the route of administration, which may be by bolus, infusion, injection or any other suitable route, as discussed below, and are well known in the art.

The cyclic peptides can be formulated into suitable delivery vehicles. For parenteral administration, e.g. by injection, the pharmaceutical composition comprising the cyclic peptide described herein may be in the form of a parenterally acceptable aqueous solution or suspension in a physiologically acceptable diluent with a suitable pharmaceutical carrier. Those of relevant skill in the art are well able to prepare suitable solutions using, suitable, carriers, preservatives, stabilizers, buffers, antioxidants and/or other additives may be employed as required. Suitable carriers, excipients, etc. can be found in standard pharmaceutical texts, for example, Remington's Pharmaceutical Sciences, 18th edition, Mack Publishing Company, Easton, Pa., 1990.

The term parenteral as used herein includes subcutaneous, intravenous, intradermal, intramuscular, intraperitoneal, and intrathecal administration of a cyclic peptide or composition described herein. A cyclic peptide or composition described herein may also be administered by nasal or gastric methods

The cyclic peptides described herein are preferably formulated and administered as a sterile solution although in some cases it may also be possible to use lyophilized preparations. Sterile solutions are prepared by sterile filtration or by other methods known in the art. The solutions are then lyophilized or filled into pharmaceutical dosage containers.

The pharmaceutical compositions may be for use as a vaccine. The vaccine composition may further comprise an adjuvant. Adjuvants are known in the art to further increase the immune response to an applied antigenic determinant. Adjuvants are defined as one or more substances that cause stimulation of the immune system. In this context, an adjuvant is used to enhance an immune response to the cyclic peptides of the invention. Examples of suitable adjuvants include but are not limited to aluminium salts such as aluminium hydroxide and/or aluminium phosphate; oil-emulsion compositions (or oil-in-water compositions), including squalene-water emulsions, such as MF59; saponin formulations, such as for example QS21 and Immunostimulating Complexes (ISCOMS); bacterial or microbial derivatives, examples of which are monophosphoryl lipid A (MPL), 3-0-deacylated MPL (3dMPL), CpG-motif containing oligonucleotides, ADP-ribosylating bacterial toxins or mutants thereof, such as E. coli heat labile enterotoxin LT, cholera toxin CT, and the like; eukaryotic proteins (e.g. antibodies or fragments thereof) which stimulate immune response upon interaction with recipient cells. In certain embodiments the compositions of the invention comprise aluminium as an adjuvant, e.g. in the form of aluminium hydroxide, aluminium phosphate, aluminium potassium phosphate, or combinations thereof.

The present invention provides cyclic peptides which mimic an epitope on AβpE3-x or Aβ4-x making them suitable for vaccination against amyloid-associated disease. The cyclic peptides as described herein are immunogenic. By immunogenic it is meant the cyclic peptide has the ability to provoke an immune response. The immunogenic compositions comprising the cyclic peptides as described herein can induce an immune response against the cyclic peptide and facilitate the generation of anti-amyloid antibodies, in particular anti-amyloid antibodies that specifically bind low molecular weight oligomers of AβpE3-x or Aβ4-x.

Without being bound by theory, it is believed that administration of the cyclic peptide as described herein as a vaccine, will induce an immune response which leads to the generation of anti-Aβ antibodies that bind specifically to low molecular weight AβpE3-x or Aβ4-x oligomers. These anti-Aβ antibodies will neutralize the toxic Aβ oligomers generated early in the pathology of Alzheimer's disease and may prevent the subsequent formation of plaques.

Accordingly, the invention provides methods for inducing an immune response against amyloid-beta in a subject comprising administering to the subject a therapeutically effective amount of a cyclic peptide according to the invention. Also provided are compositions according to the invention for use in inducing an immune response in a subject, in particular for use as a vaccine. Further provided is the use of the cyclic peptide described herein according to the invention for the manufacture of a medicament for use in inducing an immune response protein in a subject. Preferably, the induced immune response is characterized by the production of antibodies capable of binding specifically to low-molecular weight oligomers of amyloid-beta.

The invention also provides methods for the treatment of Alzheimer's Disease, in particular wherein the Alzheimer's disease is sporadic Alzheimer's disease or familial Alzheimer's disease, and other Aβ-related diseases and disorders and other neurological diseases characterised by soluble amyloids. Accordingly, the invention also relates to methods of treating Alzheimer's disease comprise administering to the subject a therapeutically effective amount of a cyclic peptide as described herein.

Treatment includes both prophylaxis and therapeutic treatment. The terms “treat”, “treating” or “treatment” (or equivalent terms) mean that the severity of the individual's condition is reduced or at least partially improved or ameliorated and/or that some alleviation, mitigation or decrease in at least one clinical symptom is achieved and/or there is an inhibition or delay in the progression of the condition and/or prevention or delay at the onset of a disease or illness

In particular, treatment of Alzheimer's disease includes, preventing or delaying the onset of Alzheimer's disease and/or one or more symptoms associated with Alzheimer's Disease in a subject. Treatment encompasses inhibiting or reducing the accumulation of amyloid-beta oligomers in the subject.

The treatment methods mentioned above may comprise administration of the antibody or composition described herein (e.g., a composition comprising a cyclic peptide described herein, a pharmaceutically acceptable excipient and optionally an additional therapeutic agent) to an individual under conditions that generate a beneficial therapeutic response in the individual e.g., for the prevention or treatment of Alzheimer's disease. Such an individual may be suffering from Alzheimer's disease. The methods of treatment described herein may be used on both asymptomatic patients, and those currently showing symptoms of Alzheimer's disease. A cyclic peptide described herein may be administered prophylactically to an individual who does not have Alzheimer's disease. A cyclic peptide described herein may be administered to an individual who does not have, or does not exhibit the symptoms of, Alzheimer's disease. A cyclic peptide described herein may be administered to an individual who does have, or appears to have, Alzheimer's disease. Individuals amenable to treatment include individuals at risk of or susceptible to Alzheimer's disease but not showing symptoms and individuals suspected of having Alzheimer's disease, as well as individuals presently showing symptoms. Cyclic peptides described herein may be administered prophylactically to the general population. In some embodiments, individuals suitable for treatment as described herein may include individuals with early onset Alzheimer's disease or one or more symptoms thereof, and individuals for whom amyloid peptide is detected in a sample of bodily fluid, such as CSF.

The terms “patient”, “individual” or “subject” include human and other mammalian subjects that receive either prophylactic or therapeutic treatment with one or more cyclic peptides described herein. Mammalian subjects include primates, e.g., non-human primates. Mammalian subjects also include laboratory animals commonly used in research, such as but not limited to, rabbits and rodents such as rats and mice.

A cyclic peptide described herein may be used in a method of preventing or treating Alzheimer's disease that involves administering to the patient an effective dosage of the cyclic peptide as described herein. As used herein, an “effective amount” or an “effective dosage” or a “sufficient amount” (or grammatically equivalent terms) of a cyclic peptide described herein refers to an amount of cyclic peptide or composition described herein that is effective to produce a desired effect, which is optionally a therapeutic effect (i.e., by administration of a therapeutically or prophylactically effective amount). For example, an “effective amount” or an “effective dosage” or a “sufficient amount” may be an amount so that the severity of the individual's condition, e.g., Alzheimer's disease, is reduced or at least partially improved or ameliorated and/or that some alleviation, mitigation or decrease in at least one clinical symptom is achieved and/or there is an inhibition or delay in the progression of Alzheimer's disease and/or prevention or delay at the onset of Alzheimer's disease.

In both prophylactic and therapeutic treatment regimes, reagents may be administered in several dosages until a sufficient immune response has been achieved. The term “immune response” or “immunological response” includes the development of a humoral (antibody mediated) and/or a cellular (mediated by antigen-specific T cells or their secretion products) response directed against an antigen in a recipient subject. Typically, the immune response is monitored and repeated dosages are given if the immune response starts to wane.

Effective doses of the compositions described herein, for the treatment of the above described conditions vary depending upon many different factors, including means of administration, target site, physiological state of the patient, whether the patient is human or an animal, other medications administered, and whether treatment is prophylactic or therapeutic.

For active immunization with a cyclic peptide described herein the dosage ranges from about 0.1 to 100 mg/kg, and more usually 0.1 to 50 mg/kg, of the host body weight. For example, dosages may be at least 1 mg/kg body weight or at least 10 mg/kg body weight or within the range of 1-100 mg/kg. In another example, dosages may be at least 0.5 mg/kg body weight or at least 50 mg/kg body weight or within the range of 0.5-50 mg/kg, preferably at least 5 mg/kg. In a preferred example, dosages may be about 50 mg/kg.

The treatments described herein may comprise the administration of the cyclic peptide to a subject as a single dose, in two doses, or in multiple doses. A cyclic peptide described herein may be administered on multiple occasions. Intervals between single dosages may be daily, weekly, monthly or yearly. Intervals may also be irregular as indicated by measuring blood levels of the anti-Aβ antibodies induced in the patient. In some methods, dosage is adjusted to achieve a desired plasma antibody concentration. Dosage and frequency vary depending on the patient.

The dosage and frequency of administration may vary depending on whether the treatment is prophylactic or therapeutic. In prophylactic applications, compositions containing the cyclic peptides described herein are administered to a patient not already in the disease state to enhance the patient's resistance. Such an amount is defined to be a “prophylactic effective dose.” In this use, the precise amounts again depend upon the patient's state of health and general immunity, but generally range from 0.1 to 25 mg per dose, especially 0.5 to 2.5 mg per dose. A relatively low dosage is administered at relatively infrequent intervals over a long period of time.

The compositions according to the invention may be used in stand-alone treatment and/or prophylaxis of a disease or condition caused by amyloid-beta proteins, i.e. a neurodegenerative disease such as Alzheimer's disease, or in combination with other prophylactic and/or therapeutic treatments, such as other vaccines, and/or antibodies, and/or other active agents. In certain embodiments, the vaccine may be a combination vaccine that further comprises other components that induce an immune response, e.g. against other proteins associated with Alzheimer's disease and/or induce antibodies directed to other forms of amyloid-beta. The administration of further active components may for instance be done by separate administration or by administering combination products of the vaccines of the invention and the further active components.

A cyclic peptide described herein may be provided in the form of a kit. Kits may contain at least one cyclic peptide described herein. A kit may comprise a composition described herein, in one or more containers, optionally with one or more other prophylactic or therapeutic agents useful for the prevention, management or treatment of Alzheimer's disease (AD). If the composition containing components for administration is not formulated for delivery via the alimentary canal, such as oral delivery, a device capable of delivering the kit components through some other route may be included, e.g., a syringe. The kit may further comprise a composition comprising other therapeutic agents for other diseases or conditions. The kit may further include instructions for preventing, treating, managing or ameliorating AD, as well as side effects and dosage information for method of administration.

The invention also relates to methods of producing a cyclic peptide as described herein. A cyclic peptide as described herein can be prepared by methods known in the art. In one embodiment the method comprises generating a linear peptide comprising the sequence of the desired peptide and cyclising the linear peptide via the cysteine residue to obtain the cyclic peptide. The linear peptides generated can be cyclised by methods known in the art, for example thiol oxidation, optionally with the introduction with a methylene bridge. See also Kourra C and Cramer N, Chem. Sci., 2016, 7, 7007-7012.

The invention also relates to methods for the production of an antibody that recognise low molecular weight oligomers of amyloid beta comprising:

(a) immunizing an animal with a cyclic peptide or variant thereof as described above, a cyclic peptide comprising the sequence of formula (I), preferably the sequence of SEQ ID NO: 14, SEQ ID NO: 4 or SEQ ID NO:13;
(b) obtaining the antibodies generated by the immunization in step (a).

The method can further comprise step (c) comprising screening the antibodies obtained in step (b). Preferably the antibodies are screened for their specific recognition of low molecular weight oligomers of amyloid beta. Preferable the antibodies are screened for their ability to specifically recognise N-terminal truncated amyloid peptides, i.e. AβpE3-x and Aβ4-x, and that do not significantly bind Aβ1-42, preferably specifically recognise AβpE3-42 and Aβ4-42.

Antibodies can be screened for their binding and/or specificity to low molecular weight oligomers of amyloid-beta, preferably low molecular weight oligomers of AβpE3-x and Aβ4-x, using standard methods known in the art, such as ELISA. For example, using assays as described in WO2011/151076 and WO2020/070225. For selection of an antibody that specifically binds a low molecular weight oligomer, but that does not specifically bind other forms of amyloid-beta protein. for example, high molecular weight oligomers and/or monomeric and dimeric forms of amyloid-beta, can be done on the basis of positive binding to the low molecular weight oligomers of amyloid-beta and the lack of binding to the high molecular weight oligomers and/or monomeric and dimeric forms of amyloid-beta. Selection of an antibody that specifically binds AβpE3-42 and Aβ4-42, but that does not specifically bind Aβ1-42, can be done on the basis of positive binding to AβpE3-42 and Aβ4-42 and the lack of binding to Aβ1-42.

Other aspects and embodiments described herein provide the aspects and embodiments described above with the term “comprising” replaced by the term “consisting of” and the aspects and embodiments described above with the term “comprising” replaced by the term “consisting essentially of”.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

Modifications of the above embodiments, further embodiments and modifications thereof will be apparent to the skilled person on reading this disclosure, and as such, these are within the scope described herein.

All documents and sequence database entries mentioned in this specification are incorporated herein by reference in their entirety for all purposes.

### EXAMPLES

### Experimental Methods

### 1. Production of Cyclized Peptide

In summary a linear peptide comprising the desired sequence and comprising two cysteine residues is generated, using standard techniques in the art. The peptide is cyclised via the cysteine residues present therein.
The peptides were cyclised by standard methods in the art, e.g. thiol oxidation, optionally with the introduction with a methylene bridge. See also for example Kourra C and Cramer N, Chem. Sci., 2016, 7, 7007-7012.
Peptides having the following sequences were generated:

| Peptide | Sequence | SEQ ID NO: |
|---|---|---|
| 3, 12 | DACFRHDSGYECHH | 4 |
| 2, 12 | DCEFRHDSGYECHH | 5 |
| 4, 12 | DAECRHDSGYECHH | 6 |
| 5, 12 | DAEFCHDSGYECHH | 7 |
| 3, 11 | DACFRHDSGYCVHH | 8 |
| 2, 11 | DCEFRHDSGYCVHH | 9 |
| 2, 10 | DCEFRHDSGCEVHH | 10 |
| 3, 10 | DACFRHDSGCEVHH | 11 |
| 2, 13 | DCEFRHDSGYEVCH | 12 |
| 3, 13 | DACFRHDSGYEVCH | 13 |
| 1, 13 | CAEFRHDSGYEVCH | 14 |
| 1, 12 | CAEFRHDSGYECHH | 15 |
| 1, 11 | CAEFRHDSGYCVHH | 16 |
| 1, 10 | CAEFRHDSGCEVHH | 17 |

The peptides were cyclised via their cysteine residues either with a disulphide bridge having the formula —S—S— or a thioacetal bridge having the formula —S—CH₂—S—.

### 2. 1-14 Disulphide Bridged Cyclic Peptide Binding ELISA

1. Coat 384-well plate with 30 μL/well of 2.5 μg/ml streptavidin (Thermo Scientific 21122) diluted in PBS (Thermo Fisher 10010-015)
2. Incubate at 4° C. overnight
3. Wash the plate (NUNC 384 program)
4. Coat 384-well plate with 30 μL/well of 2 μg/ml disulphide bridged cyclic peptide diluted in PBS
5. Incubate at RT for 1 hour
6. Wash the plate (NUNC 384 program)
7. Block the plate with 80 μL/well of assay buffer
8. Incubate at 4° C. overnight
9. Wash the plate (NUNC 384 program)
10. For sera: Dispense 70 μL/well of sera samples (diluted 1/100 in assay buffer) on a non-sticky plate and dilute in a 2-fold series in assay buffer (35 μL into 35 μL assay buffer)
   For competitor antibodies: Dispense 60 μL/well of control antibodies (diluted to 100.0 μg/ml in assay buffer) on a non-sticky plate and dilute in a 3-fold series in assay buffer (20 μL into 40 μL assay buffer)
11. Dispense 60 μL/well of control antibodies (diluted to 360.0 μg/ml in assay buffer) on a non-sticky plate and dilute in a 3-fold series in assay buffer (20 μL into 40 μL assay buffer)
12. Transfer 30 μL/well onto the assay plate
13. Incubate at 37° C. for 1 hour
14. Wash the plate (NUNC 384 program)
15. Dilute the secondary antibody appropriately in assay buffer and add 30 μL/well
16. Incubate at 37° C. for 1 hour
17. Wash the plate (NUNC 384 program)
18. Add 20 μL/well of K-BLUE substrate (Neogen 308176)
19. Incubate at RT for 10 min in the dark
20. Stop the reaction by adding 10 μL/well of RED STOP solution (Neogen 308176)
21. Read the optical density at 650 nm using the PheraStar Plus (BMG LabTech)

### 3. 1-42 Peptide Binding ELISA

1. Coat 384-well plate with 30 μL/well of 100 ng/ml PSL amyloid 1-42 peptide (Human-Peptide Specialty Laboratories-CEM1904161) diluted in carb/bicarb buffer
2. Incubate at 37° C. for 1 hour
3. Wash the plate (NUNC 384 program)
4. Block the plate with 80 μL/well of assay buffer
5. Incubate at 4° C. overnight
6. Wash the plate (NUNC 384 program)
7. For disulphide bridged immunisation sera:
   Dispense 70 μL/well of samples (diluted 1/100 in assay buffer) on a non-sticky plate and dilute in a 2-fold series in assay buffer (35 μL into 35 μL assay buffer)
   For thioacetal bridged immunisation sera:
   Dispense 60 μL/well of sera samples (diluted 1/100 in assay buffer) and control antibodies (diluted to 360.0 μg/ml in assay buffer) on a non-sticky plate and dilute in a 3-fold series in assay buffer (20 μL into 40 μL assay buffer)
8. For control antibodies: Dispense 60 μL/well of control antibodies (diluted to 360.0 μg/ml in assay buffer) on a non-sticky plate and dilute in a 2 or 3-fold series (dependent on dilutions of immunisation sera) in assay buffer (20 μL into 40 μL assay buffer)
9. Transfer 30 μL/well onto the assay plate
10. Incubate at 37° C. for 1 hour
11. Wash the plate (NUNC 384 program)
12. Dilute the secondary antibody appropriately in assay buffer and add 30 μL/well
13. Incubate at 37° C. for 1 hour
14. Wash the plate (NUNC 384 program)
15. Add 20 μL/well of K-BLUE substrate (Neogen 308176)
16. Incubate at RT for 10 min in the dark
17. Stop the reaction by adding 10 μL/well of RED STOP solution (Neogen 308176)
18. Read the optical density at 650 nm using the PheraStar Plus (BMG LabTech)
   4. pE3-42 Peptide Binding ELISA
1. Coat 384-well plate with 30 μL/well of 100 ng/ml PSL amyloid pE3-42 peptide (Human-Peptide Specialty Laboratories-CEM062210 Pyr) diluted in carb/bicarb buffer
2. Incubate at 37° C. for 1 hour
3. Wash the plate (NUNC 384 program)
4. Block the plate with 80 μL/well of assay buffer
5. Incubate at 4° C. overnight
6. Wash the plate (NUNC 384 program)
7. For disulphide bridged immunisation sera:
   Dispense 70 μL/well of sera samples (diluted 1/100 in assay buffer) on a non-sticky plate and dilute in a 2-fold series in assay buffer (35 μL into 35 μL assay buffer)
For thioacetal bridged immunisation sera:
   Dispense 60 μL/well of sera samples (diluted 1/100 in assay buffer) and control antibodies (diluted to 360.0 μg/ml in assay buffer) on a non-sticky plate and dilute in a 3-fold series in assay buffer (20 μL into 40 μL assay buffer)
8. For control antibodies: Dispense 60 μL/well of control antibodies (diluted to 360.0 μg/ml in assay buffer) on a non-sticky plate and dilute in a 2 or 3-fold series (dependent on dilutions of immunisation sera) in assay buffer (20 μL into 40 μL assay buffer)
9. Transfer 30 μL/well onto the assay plate
10. Incubate at 37° C. for 1 hour
11. Wash the plate (NUNC 384 program)
12. Dilute the secondary antibody appropriately in assay buffer and add 30 μL/well
13. Incubate at 37° C. for 1 hour
14. Wash the plate (NUNC 384 program)
15. Add 20 μL/well of K-BLUE substrate (Neogen 308176)
16. Incubate at RT for 10 min in the dark
17. Stop the reaction by adding 10 μL/well of RED STOP solution (Neogen 308176)
18. Read the optical density at 650 nm using the PheraStar Plus (BMG LabTech)

### 5. 4-42 Peptide Binding ELISA

1. Coat 384-well plate with 30 μL/well of 200 ng/ml Anaspec 4-42 peptide (Eurogentec AS-29908-1) diluted in carb/bicarb buffer
2. Incubate at 37° C. for 1 hour
3. Wash the plate (NUNC 384 program)
4. Block the plate with 80 μL/well of assay buffer
5. Incubate at 4° C. overnight
6. Wash the plate (NUNC 384 program)
7. For disulphide bridged immunisation sera:
   Dispense 70 μL/well of sera samples (diluted 1/100 in assay buffer) on a non-sticky plate and dilute in a 2-fold series in assay buffer (35 μL into 35 μL assay buffer)
For thioacetal bridged immunisation sera:
   Dispense 60 μL/well of control antibodies (diluted to 360.0 μg/ml in assay buffer) on a non-sticky plate and dilute in a 3-fold series in assay buffer (20 μL into 40 μL assay buffer)
8. Transfer 30 μL/well onto the assay plate
9. Incubate at 37° C. for 1 hour
10. Wash the plate (NUNC 384 program)
11. Dilute the secondary antibody appropriately in assay buffer and add 30 μL/well
12. Incubate at 37° C. for 1 hour
13. Wash the plate (NUNC 384 program)
14. Add 20 μL/well of K-BLUE substrate (Neogen 308176)
15. Incubate at RT for 10 min in the dark
16. Stop the reaction by adding 10 μL/well of RED STOP solution (Neogen 308176)
17. Read the optical density at 650 nm using the PheraStar Plus (BMG LabTech)

### 6. 8.5. KLH Antigen Binding ELISA

1. Coat 384-well plate with 30 μL/well of 2 μg/ml KLH (Sigma H8283) diluted in PBS (Thermo Fisher 10010-015)
2. Incubate at 4° C. overnight
3. Wash the plate (NUNC 384 program)
4. Block the plate with 80 μL/well of assay buffer
5. Incubate at RT for 1 hour
6. Wash the plate (NUNC 384 program)
7. Dispense 60 μL/well of sera samples (diluted 1/1000 in assay buffer) and control antibodies (diluted to 20.0 μg/ml in assay buffer) on a non-sticky plate and dilute in a 3-fold series in assay buffer (20 μL into 40 μL assay buffer)
8. Transfer 30 μL/well onto the assay plate
9. Incubate at 37° C. for 1 hour
10. Wash the plate (NUNC 384 program)
11. Dilute the secondary antibody appropriately in assay buffer and add 30 μL/well
12. Incubate at 37° C. for 1 hour
13. Wash the plate (NUNC 384 program)
14. Add 20 μL/well of K-BLUE substrate (Neogen 308176)
15. Incubate at RT for 5 min in the dark
16. Stop the reaction by adding 10 μL/well of RED STOP solution (Neogen 308176)
17. Read the optical density at 650 nm using the PheraStar Plus (BMG LabTech)

### 7. Thioacetal Bridged Cyclic Peptide Binding ELISA

1. Coat 384-well plate with 30 μL/well of 2.5 μg/ml streptavidin (Thermo Scientific 21122) diluted in PBS (Thermo Fisher 10010-015)
2. Incubate at 4° C. overnight
3. Wash the plate (NUNC 384 program)
4. Coat 384-well plate with 30 μL/well of 2 μg/ml thioacetal bridged cyclic peptide diluted in PBS
5. Incubate at RT for 1 hour
6. Wash the plate (NUNC 384 program)
7. Block the plate with 80 μL/well of assay buffer
8. Incubate at 4° C. overnight
9. Wash the plate (NUNC 384 program)
10. Dispense 60 μL/well of sera samples (diluted 1/100 in assay buffer) and control antibodies (diluted to 360.0 μg/ml in assay buffer) on a non-sticky plate and dilute in a 3-fold series in assay buffer (20 μL into 40 μL assay buffer)
11. Transfer 30 μL/well onto the assay plate
12. Incubate at 37° C. for 1 hour
13. Wash the plate (NUNC 384 program)
14. Dilute the secondary antibody appropriately in assay buffer and add 30 μL/well
15. Incubate at 37° C. for 1 hour
   16. Wash the plate (NUNC 384 program)
   17. Add 20 μL/well of K-BLUE substrate (Neogen 308176)
   18. Incubate at RT for 10 min in the dark
   19. Stop the reaction by adding 10 μL/well of RED STOP solution (Neogen 308176)
   20. Read the optical density at 650 nm using the PheraStar Plus (BMG LabTech)

### 8. Protein Expression and Purification for Crystallography Studies

The Fab fragments for anti-β-amyloid Fabs TAP01 and TAP01_01 were expressed in Expi293 cells. The pE3-14 and cyclised 3-14 peptides were solubilised in 25 mM Tris-HCl (pH 7.5) and 50 mM NaCl to 1 mM. Fab/peptide complexes were typically mixed at 1:1.5 molar ratio in 25 mM Tris-HCl (pH 7.5) and 50 mM NaCl. For crystallisation, all Fab/peptide complex samples were concentrated to ˜14 mg/ml.

### 9. Crystallization, Structure Determination, and Refinement

All crystals were obtained by the vapor diffusion method at 19° C., by mixing equal volumes of protein plus well solution.

The TAP01-pE3-14 crystals grew in 20% PEG3350 and 0.2 M ammonium citrate. TAP01_01-pE3-14 crystals grew in 10% PEG 20K, 20% PEG550MME, 0.1 M MOPS/HEPES, pH 7.5, and 0.03 M each of sodium nitrate, disodium hydrogen phosphate and ammonium sulphate.

TAP01-cyclised 3-14 co-crystals grew in 20% PEG 6K, 0.1M HEPES, pH 7.0, and 0.01 M zinc chloride. For cryoprotection, crystals were generally transferred to a solution of mother liquor plus 22% ethylene glycol.

Data sets were collected at the European Synchrotron Radiation Facility (beamline ID30B (TAP01+pE3-14)) or at Diamond Light Source (beamline 104 (TAP01_01+pE3-14 and TAP01+cyclised 3-14)). Co-crystals of TAP01 and TAP01_01 with the pE3-14 peptide were refined to 1.4 and 2.5 Å resolution, respectively, whereas TAP01 with the cyclised 3-14 peptide diffracted 2.1 Å. Data were processed using XDS (Kabsch, W. (2010a/b) Acta Cryst D66, 125-132) and AIMLESS from the CCP4 Suite (Winn, M., et al. (2011) Acta Cryst D67, 235-242).

All crystal structures were solved by molecular replacement using Phaser (McCoy et al., (2005) Acta Cryst D 61, 458-64). The TAP01 structure was solved using a homology model generated using SWISSMODEL (Waterhouse, A., et al. (2018) Nucleic Acids res. 46 W296-W303) with the deposited antibody structures 4F33 (Ma, J., et al. (2012) JBC, 287: 33123-33131) and 117Z (Larsen N. A., et al. (2001) JMB, 311: 9-15), used to model the heavy and light chains, respectively. The refined coordinates of the TAP01 structure served as the search model for the subsequent TAP01_01 structure. Atomic models were built using Coot (Emsley, P. & Cowtan, K. Coot, (2004) Acta Cryst D60, 2126-32) and refined with Refmac (Murshudov, et al., (1997) Acta Cryst D53, 240-255). All structures were solved by molecular replacement and are reported with final Rwork/Rfree values below 20/25% with good stereochemistry (Table 1).

TABLE 1

| | TAP01 + pE3-14 | | TAP01_01 + pE3-14 | | TAP01 + cyclic pep | |
|---|---|---|---|---|---|---|
| Data Collection | | | | | | |
| Beamline | ESRF ID30B | | DLS I04 | | DLS I04 | |
| Wavelength | 0.96861 | | 0.97950 | | 0.97949 | |
| Space Group | P4₁2₁2 | | P1 | | P2₁ | |
| Cell Dimensions | 71, 71, 173, 90, | | 69, 81, 95, 89, 90, 68 | | 81, 47, 124, | |
| a, b, c (Å), α, | 90, 90 | | | | 90, 91, 90 | |
| β, γ (°) | | | | | | |
| Resolution (Å) | 55.01-1.40 | | 95.02-2.50 | | 124.45-1.87 | |
| Rmerge | 0.086 | (0.682) | 0.234 | (0.981) | 0.096 | (0.767) |
| CC_{1/2} | 0.986 | (0.646) | 0.956 | (0.718) | 0.997 | (0.337) |
| I/σI | 10.3 | (2.3) | 3.2 | (1.2) | 4.5 | (0.2) |
| Completeness (%) | 99.6 | (99.9) | 100.0 | (100.0) | 99.3 | (99.6) |
| Redundancy | 7.9 | (7.9) | 3.3 | (3.3) | 3.3 | (2.6) |
| Refinement | | | | | | |
| No. of Reflections | 694016 | | 220078 | | 259824 | |
| No. of Unique | 88123 | | 66418 | | 78619 | |
| Rfactor/Rfree (%) | 19.0 | (24.1) | 28.1 | (33.0) | 28.0 | (34.0) |

| | | | |
|---|---|---|---|
| Wilson B-factors (Å) | 24.0 | 25.9 | 33.0 |
| B-factors (Å) | | | |
| Protein | 21.6 | 22.3 | 37.5 |
| R.M.S. Deviations | | | |
| Bond lengths (Å) | 0.024 | 0.012 | 0.010 |
| Bond angles (°) | 2.268 | 1.658 | 1.794 |

### Results and Discussion

### 1. Identification of a Novel Epitope and Generation of ‘Constrained’ Cyclic Peptide

A novel epitope of amyloid peptides for the TAP01 antibodies, TAP01 and TAP01_01 (also known as NT4X and NT4X_SA) have been identified. X-ray crystallographic studies were performed using the mouse TAP01 antibody and the humanised TAP01_01 antibodies, in the presence or absence of the pE3-14 peptide (Table 1).

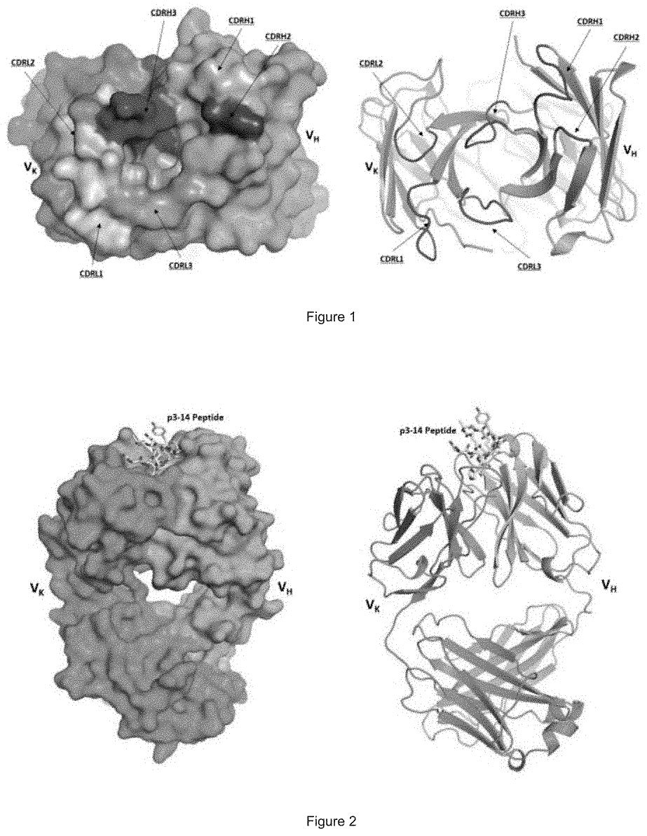

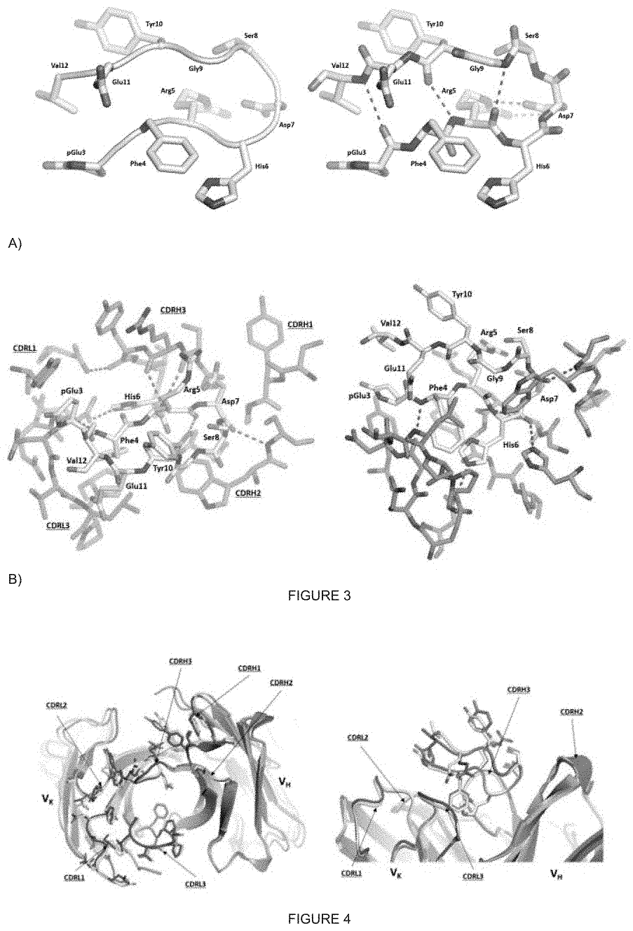

The structure of the TAP01 Fab alone (FIG. 1) and in the presence of the pE3-14 peptide (FIG. 2) were determined. These studies have shown that the TAP01 antibody binds to a hairpin structure (FIG. 3) of amyloid peptides. This binding site for the antibody had not been previously identified.

The results also show the apo structure is the same as the antibody-peptide structure, thereby demonstrating that a conformational change does not occur when the amyloid peptides are bound. In addition the structure of the TAP01 antibody and epitope are maintained during the humanisation process of the TAP01 antibody (FIG. 4).

An 1-14 amyloid peptide with cysteine residues at positions 3 and 12 forming a ‘constrained’ form of the cyclic peptide (Table 2) was generated.

Two different structures for constraining the cyclic peptides were generated: a disulphide bridged peptide and a thioacetal bridged peptide. The sequence and structure of the peptide is shown below in Table 2. The thioacetal bridged peptide provide a more chemically stable analogue of the disulphide bridged cyclic peptide. Analysis of the cyclic peptides having the sequence DACFRHDSGYECHH showed the cyclic peptides mimic the hairpin structure identified in the structural studies.

TABLE 2

| Name | peptide sequence | peptide structure |
|---|---|---|
| 1-14 cyclic peptide (disulphide bridged) | DA*CFRHDSGYE*CHH-Biotin (*S—S* bridge) | 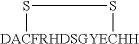 |
| 1-14 cyclic peptide (thioacetal bridged) | DAC*FRHDSGYEC*HH-[Cys]- amide (*S—CH2—S* bridged) | 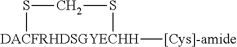 |

The X-ray crystallography studies confirmed that this ‘cyclic’ conformation could be generated and that the TAP01 antibody bound to the cyclic peptides in a similar manner as the pE3-42 peptide.

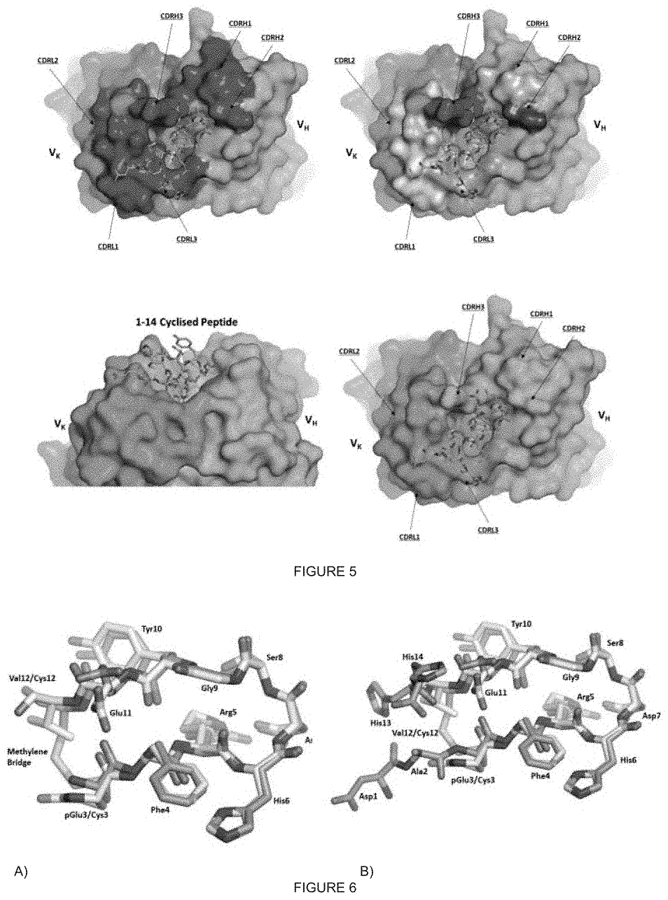

Both cyclic peptide structures revealed similar binding modes and conformations as the original structures (FIG. 4). It was also shown that the cyclic peptides adopt the same hairpin conformation as the epitope of the native pE3-14 peptide (FIGS. 5 and 6).

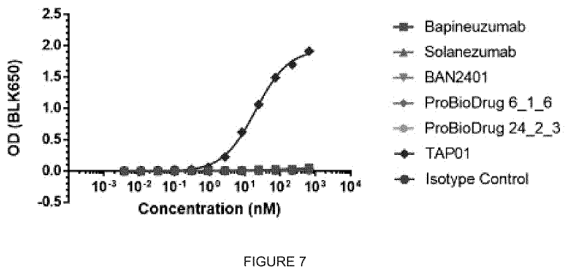

Although a number of comparator antibodies are able to bind to the pE3-42 amyloid peptide, the results show that TAP01 is the only antibody able to bind this novel hairpin epitope (FIG. 7). Binding of comparator antibodies (Bapineuzumab, solanezumab, BAN2401, ProBioDrug 6_1_6, ProBioDrug 24_2_3) to the epitope identified was investigated by ELISA using the 1-14 thioacetal bridged cyclic peptide constrained in the epitope confirmation. ProBioDrug 6_1_6 (Deposit No. DSM ACC 2924) and ProBioDrug 24_2_3 (Deposit No. DSM ACC 2926) are described in WO 20W/009987. None of the comparator antibodies tested were able to bind to this ‘cyclic’ peptide conformation.

2. Immunisation of Mice and Rabbits with Cyclic Peptides

2.1. Immunisation with Disulphide Bridged Peptide and Binding to Amyloid Peptides

Immunisation studies were performed in rabbits and mice using a 1-14 amyloid peptide sequence with cysteine residues at positions 3 and 12 and having a disulphide bridge, to investigate the potential of a vaccine approach for the treatment of AD. Animals (5 mice, 2 rabbits) were immunised with disulphide bridged cyclic peptide and sera collected at pre-immunisation (day 1), intermediate (day 35) and final time points (Day 63) as set out in Table 3.

TABLE 3

| | Day |
|---|---|
| Preimmune bleed | 1 |
| 1. Immunisation | 1 |
| 2. Immunisation | 14 |
| 3. Immunisation | 28 |
| Test bleed for ELISA | 35 |
| determination | |
| 4. Immunisation | 42 |
| 5. Immunisation | 56 |
| Final bleed | 63 |

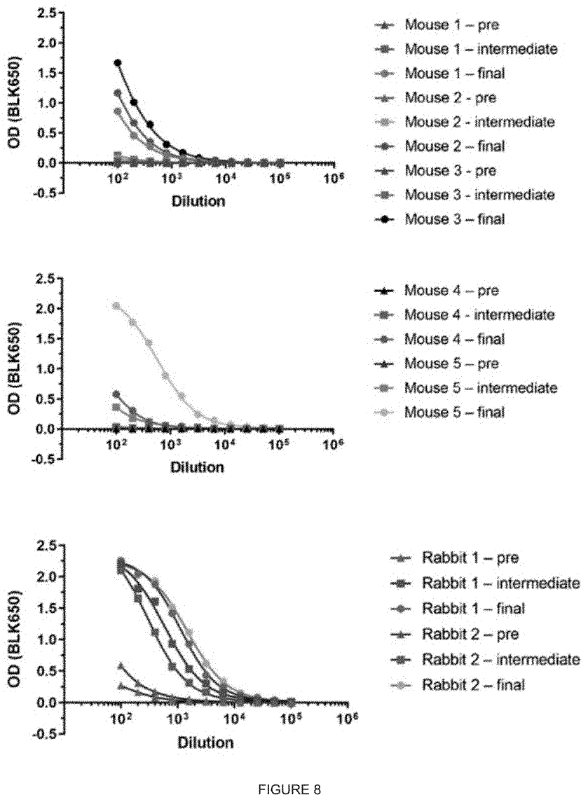

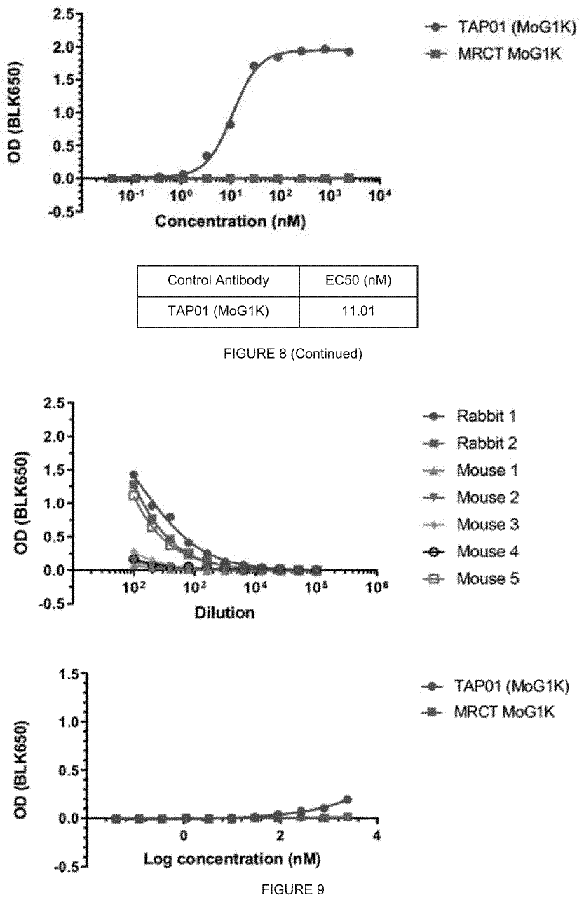

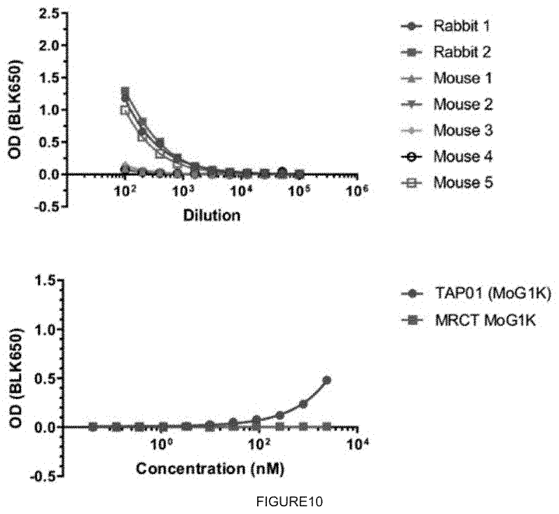

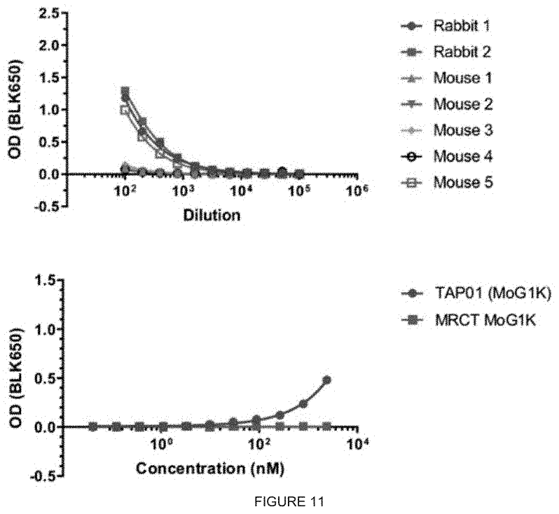

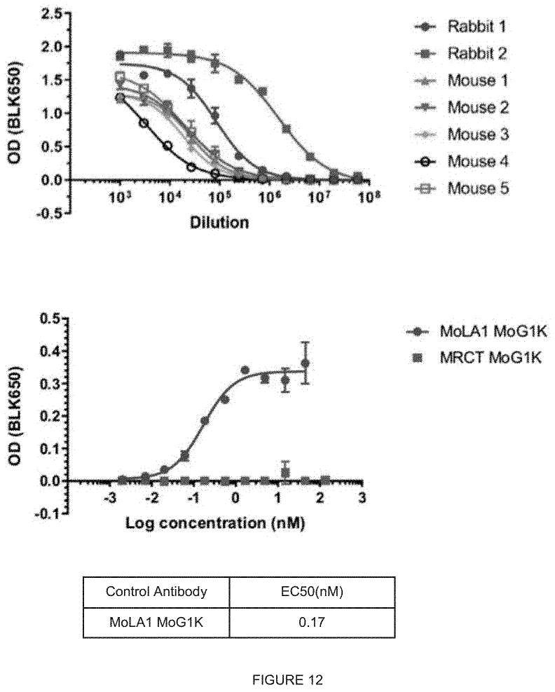

Sera were screened for binding to biotinylated cyclic, 1-42, pE3-42 and 4-42 amyloid peptides. The results are shown in FIGS. 8-12.

Results indicated that mouse 5 produced the best immune response, producing a titre of 1/3200 to the disulphide bridged cyclic peptide (FIG. 8). A higher level of background binding (pre-immunisation) was generated by the rabbits to the disulphide bridged cyclic peptide (FIG. 8). Binding of the resultant sera to the ‘cyclic’ peptide as well as to the 1-42, 4-42 and pE3-42 amyloid peptides was investigated. Minimal binding to the 4-42 and pE3-42 amyloid peptides was observed by ELISA (FIG. 9-11).

2.2. Immunisation with Thioacetal Bridged Peptide and Binding to Amyloid Peptides

Immunisation studies were performed in rabbits and mice using a 1-14 amyloid peptide sequence with cysteine residues at positions 3 and 12 and having a Thioacetal bridge, to investigate the potential of a vaccine approach for the treatment of AD. Animals were immunised with thioacetal bridged cyclic peptide and sera collected at pre-immunisation (day 1), intermediate (day 35) and final time points (Day 63) as set out in Table 3.

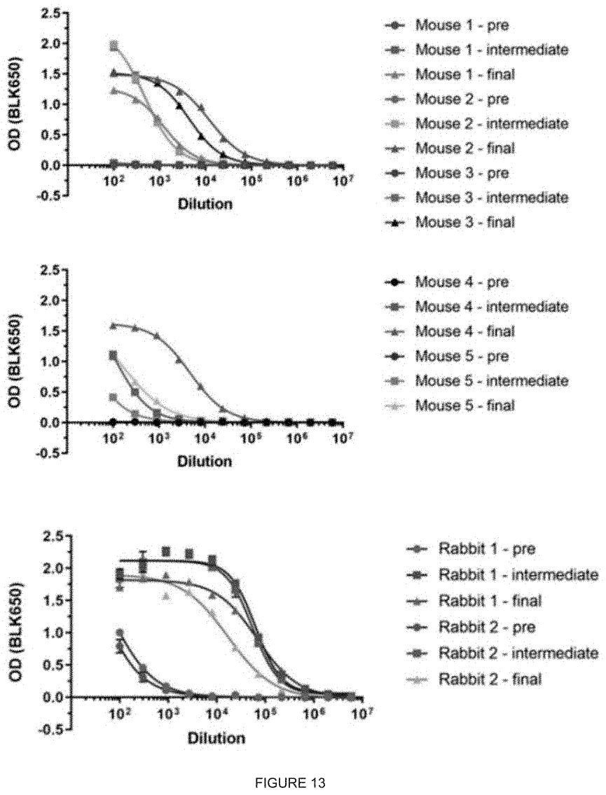

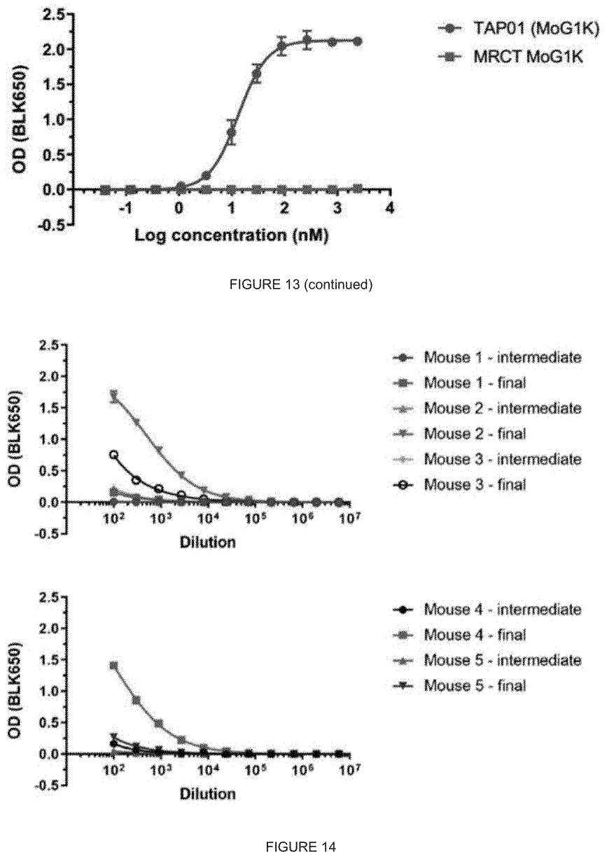

Following immunisation with the thioacetal bridged cyclic peptide (Table 3), an immune response was generated in both rabbits and mice, with higher titres obtained in mice (FIG. 13).

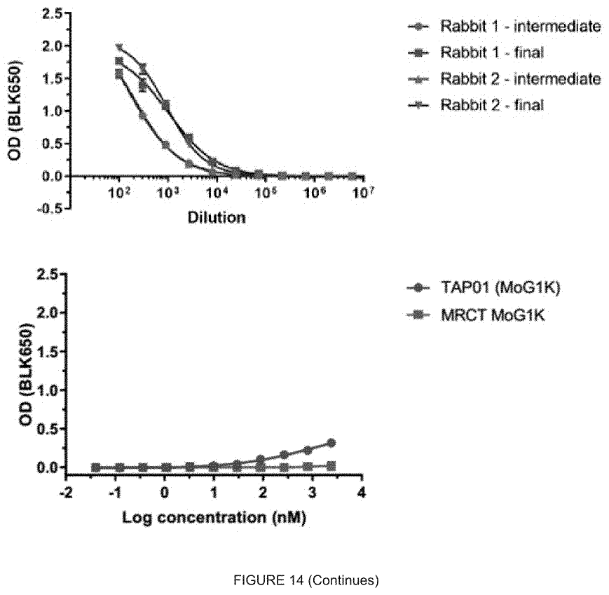

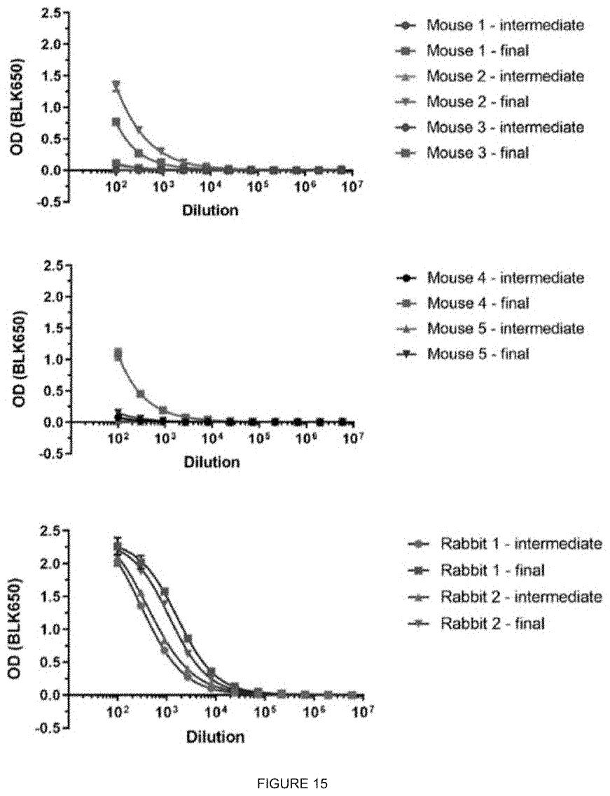

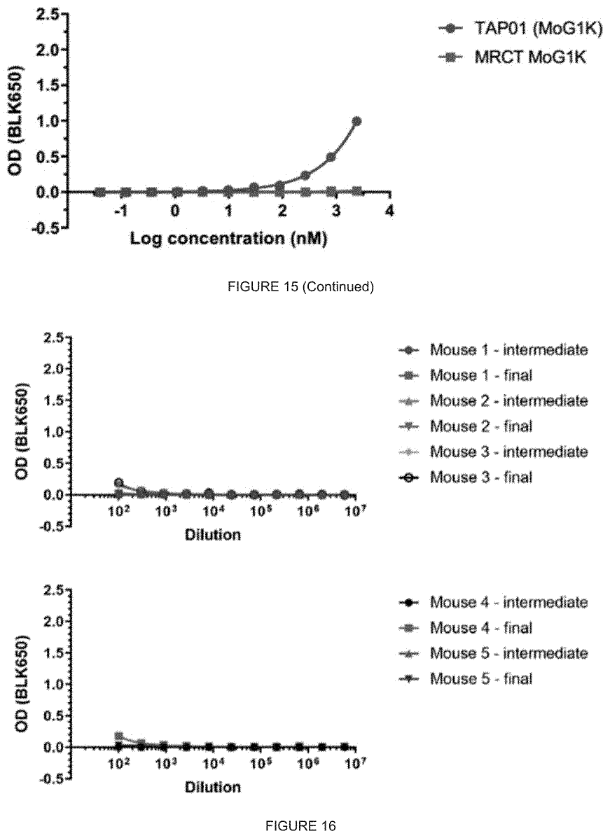

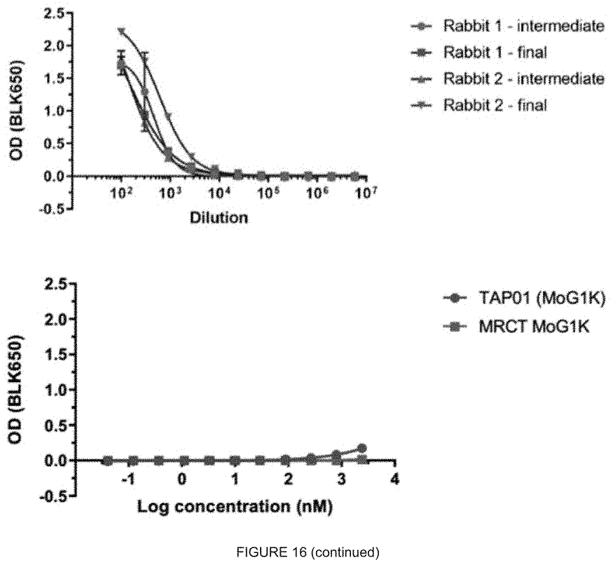

Binding of the resultant sera to the ‘cyclic’ peptide as well as to the 1-42, 4-42 and pE3-42 amyloid peptides was investigated (FIGS. 13-16). Results indicate that mice 2,3 and 4 generated the best immune responses, with titres of 1/72900 (mouse 2) and 1/24300 (mice 3 and 4) respectively to thioacetal bridged cyclic peptide (FIG. 13). In agreement with results generated following immunisation with the disulphide bridged cyclic peptide, a higher level of background binding was observed in the rabbits.

Results of testing both versions of the ‘constrained’ cyclic peptide indicated that the thioacetal bridged peptide was both more stable and generated responses with higher titres in mice. Therefore, the thioacetal bridged peptide was used in downstream experiments.

### 3. Screening Sera in Human AD Brain and 5X FAD and Tg4-42 Brain Sections

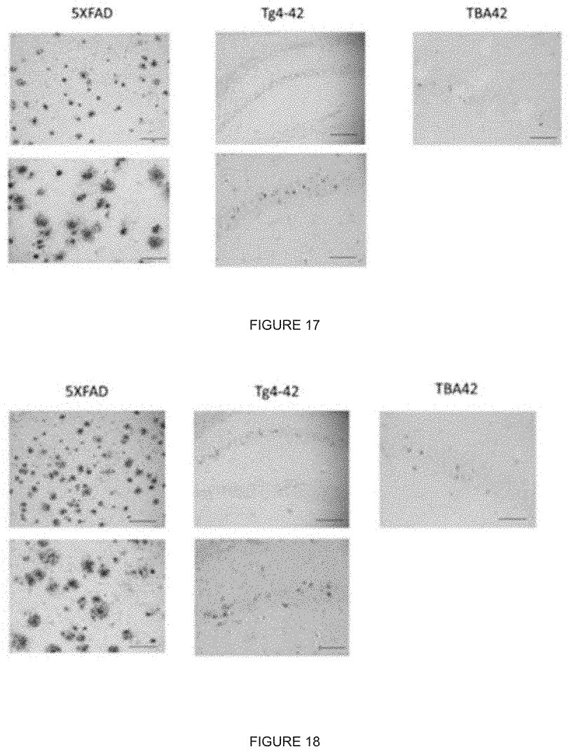

Sera from the mouse immunisations (M2 and M4 sera) were used for staining human AD brain sections and brain sections from the 5X FAD and Tg4-42 mouse models (FIGS. 17 and 18).

### 4. Biomarker Identification and Effect of TAP01 Antibodies on Glucose Metabolism

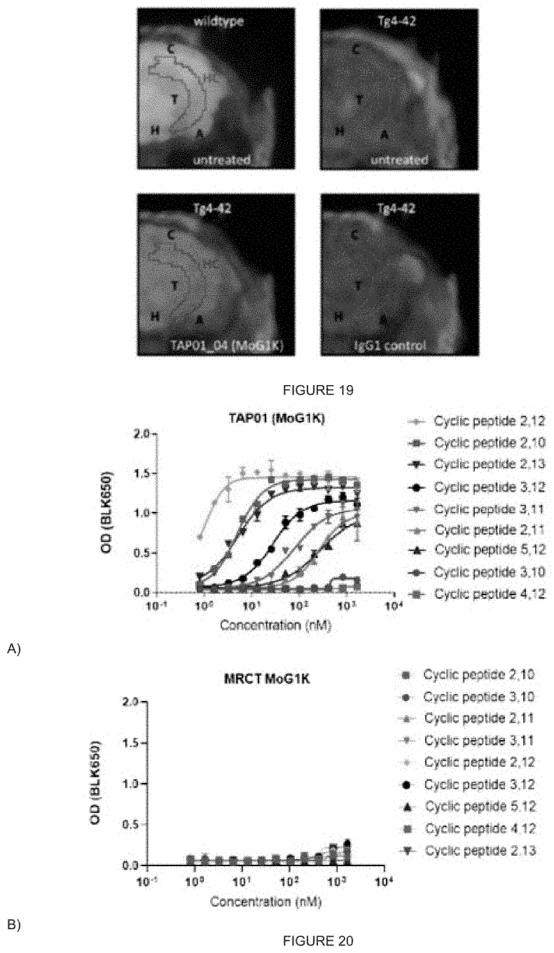

Imaging of 18F-FDG uptake in young and aged Tg4-42 mice showed decreased Cerebral Glucose Metabolism in Tg4-42 aged mice (FIG. 19). Results indicate that this reduction in cerebral glucose metabolism can be rescued with the TAP01 humanised antibody.

### 5. Generation and Assessment of Binding of TAP01_04 Antibody to 1-14 Cyclic Peptide (Thioacetal Bridged) Variants

The 1-14 thioacetal bridged cyclic peptide assessed in the above experiments has cysteine residues at positions 3 and 12 for the thioacetal bridge to constrain the peptide. In order to assess the role positioning the cysteine residues at positions 3 and 12 has for binding of the TAP01 antibody, additional peptides were generated with cysteine residues at different positions within the peptide sequence (Table 4).

TABLE 4

| Peptide | Sequence |
|---|---|
| 3, 12 | DACFRHDSGYECHH-Biotin |
| 2, 12 | DCEFRHDSGYECHH-Biotin |
| 4, 12 | DAECRHDSGYECHH-Biotin |
| 5, 12 | DAEFCHDSGYECHH-Biotin |
| 3, 11 | DACFRHDSGYCVHH-Biotin |
| 2, 11 | DCEFRHDSGYCVHH-Biotin |
| 2, 10 | DCEFRHDSGCEVHH-Biotin |
| 3, 10 | DACFRHDSGCEVHH-Biotin |
| 2, 13 | DCEFRHDSGYEVCH-Biotin |

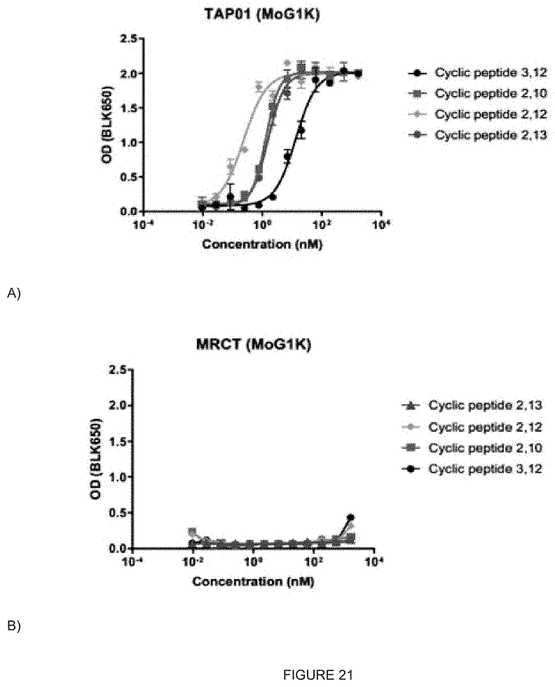

Binding of the TAP01 antibody has been assessed to these thioacetal bridged cyclic peptide variants by ELISA (FIGS. 20-22 and Table 5). Binding to comparators antibodies was also assessed.

TABLE 5

| Peptide | EC50 (nM) |
|---|---|
| 2, 10 | 1.33 |
| 2, 11 | 260.6 |
| 2, 12 | 0.24 |
| 2, 13 | 1.56 |
| 3, 10 | ND |
| 3, 11 | 79.66 |
| 3, 12 | 13.33 |
| 4, 12 | ND |
| 5, 12 | ND |

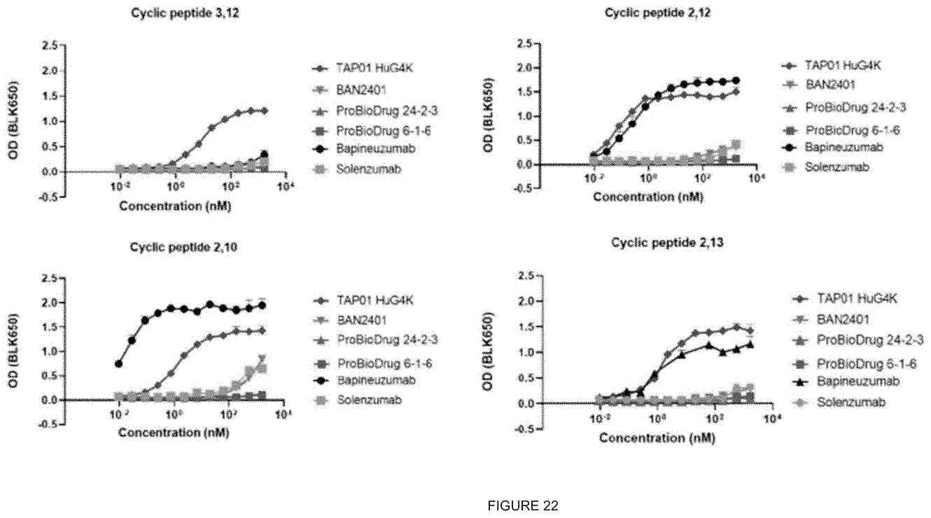

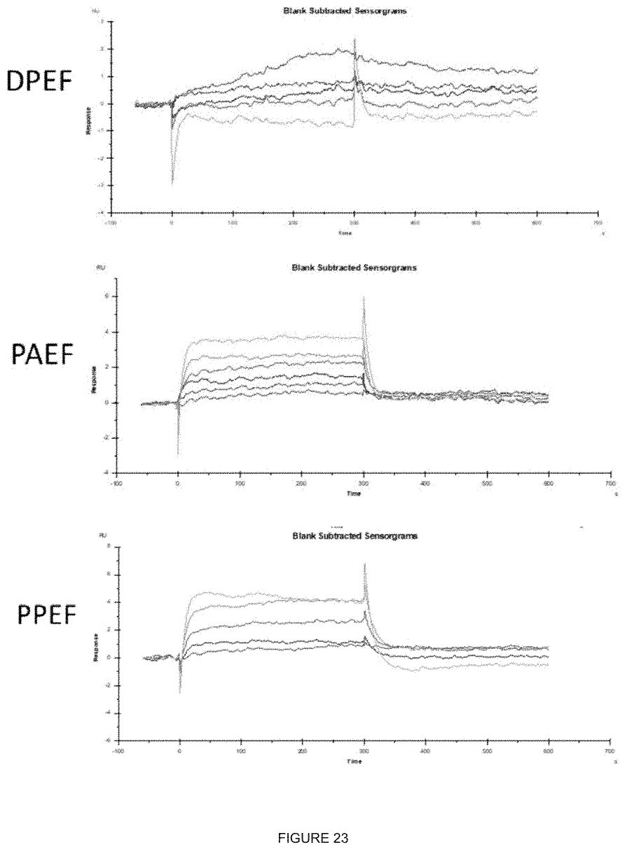

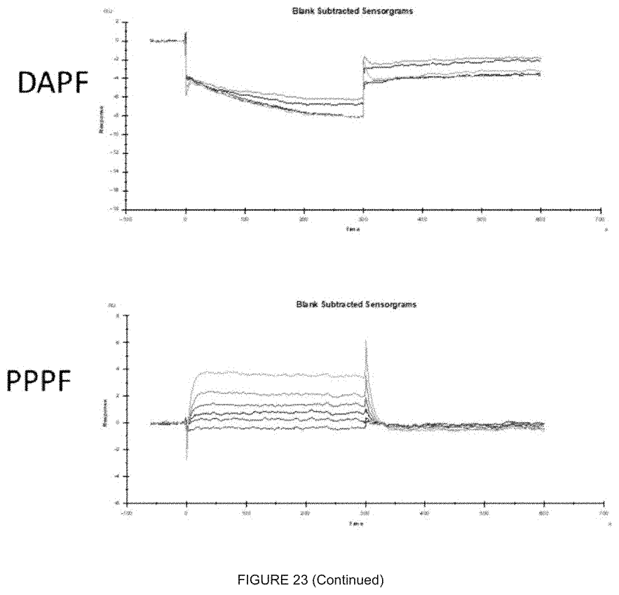

Results indicate that the affinity of the TAP01 (MoG1K) antibody is higher for the cyclic peptides 2,10, 2,12 and 2,13 compared to 3,12 (FIGS. 22 and 23), with calculated EC50 values of 1.33, 0.24 and 1.56 nM respectively, compared to 13.33 nM for 3,12. However, the comparator antibody, Bapineuzumab, is also able to bind to the 2,10, 2,12 and 2,13 cyclic peptide variants (FIG. 22). Furthermore, BAN2401 and Solanezumab are also able to bind to the 2,10 peptide variant with low affinity (FIG. 22). Therefore, this suggests that the novel hairpin epitope recognised by the TAP01 antibody is predominantly in the 3,12 confirmation.

In order to assess the role positioning the cysteine residues at different combinations of positions has for binding of the TAP01 antibody, in particular when a cysteine is provided at position 1, additional peptides were generated with cysteine residues at different positions within the peptide sequence (Table 6).

TABLE 6

| Peptide | Sequence |
|---|---|
| 3, 13 | DACFRHDSGYEVCH-Biotin |
| 1, 13 | CAEFRHDSGYEVCH-Biotin |
| 1, 12 | CAEFRHDSGYECHH-Biotin |
| 1, 11 | CAEFRHDSGYCVHH-Biotin |
| 1, 10 | CAEFRHDSGCEVHH-Biotin |

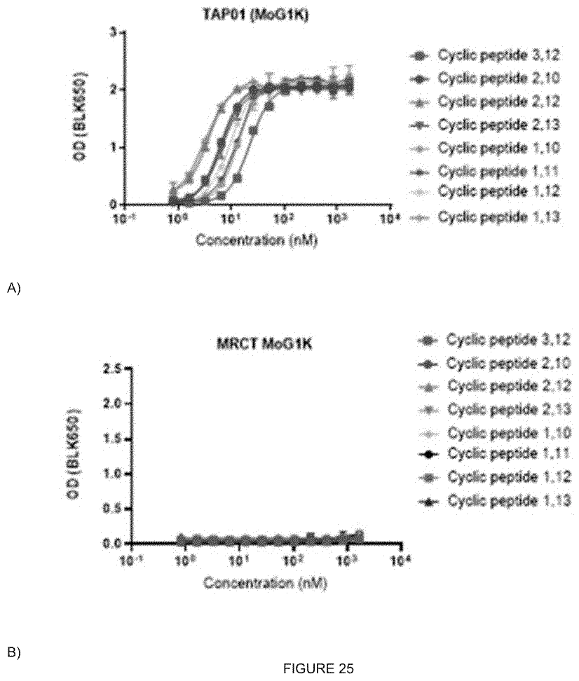

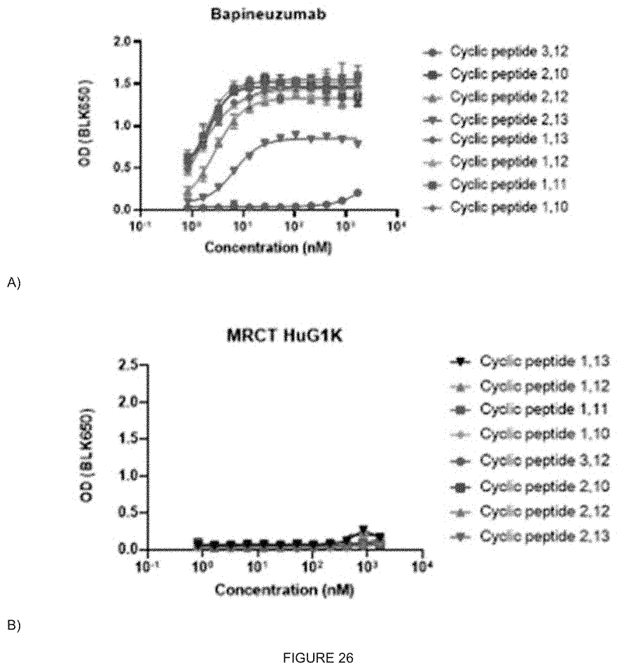

Binding of the TAP01 antibody has been assessed to these thioacetal bridged cyclic peptide variants and to cyclic peptide 3, 12, cyclic peptide 2, 10, cyclic peptide 2, 12, and cyclic peptide 2, 13 by ELISA (FIG. 25, Table 7). Binding to comparator antibodies was also assessed (FIG. 26).

TABLE 7

| Peptide | EC50 (nM) |
|---|---|
| 1, 10 | 12 |
| 1, 11 | 111 |
| 1, 12 | 9.5 |
| 1, 13 | 3.5 |
| 3, 13 | 5.414 |

Results indicate that the affinity of the TAP01 (MoG1K) antibody is highest for the cyclic peptide 1, 13 compared to cyclic peptide 1, 10, cyclic peptide 1,11, and cyclic peptide 1,12 (FIG. 25) with a calculated EC50 value of 3.5, compared to 12, 111, and 9.5 respectively.

No binding of the cyclic peptide 3,12 to the comparator antibody Bapineuzumab was seen (FIG. 26). Additionally, no binding of the cyclic peptide 3,13 to the comparator antibody Bapineuzumab was seen (FIG. 34). Binding to the comparator antibody Bapineuzumab was seen for cyclic peptide 2, 10, cyclic peptide 2, 12, cyclic peptide 2, 13, cyclic peptide 1, 10, cyclic peptide 1,11, cyclic peptide 1,12 and cyclic peptide 1,13 (FIG. 26A). However, this data further suggests that the novel hairpin epitope recognised by the TAP01 antibody is predominantly in the 3,12 conformation and also suggests that the cyclic peptide in the 3,13 conformation mimics this hairpin epitope.

### 6. Generation and Assessment of Binding of TAP01 Antibody to 1-14 Mutant Peptide Variants

To determine the mechanism of action of the amyloid peptides to the TAP01 antibody, five peptides whereby proline residues substituted the actual amino acids found in the peptide (Table 8) were generated and binding of these peptides to the TAP01 antibody was investigated (FIG. 23).

There was no binding observed with the DPEFRHDSGYEVHH and DAPFRHDSGYEVHH peptides suggesting that residues 2 (A) and 3 (E) are important for binding. Peptides, PAEFRHDSGYEVHH, PPPFRHDSGYEVHH and PPEFRHDSGYEVHH bind in a dose dependant manner suggesting residue 1 (D), a combination of residues 1, 2 and 3 (DAE) and a combination of residues 1 and 2 (DA) are not essential for binding.

TABLE 8

| Proline substitution in | | |
|---|---|---|
| peptide | Sequence | SEQ ID NO: |
| Residue 1 (D to P) | PAEFRHDSGYEVHH | 22 |
| Residue 2 (A to P) | DPEFRHDSGYEVHH | 23 |
| Residue 3 (E to P) | DAPFRHDSGYEVHH | 24 |
| Residue 1 and 2 | PPEFRHDSGYEVHH | 25 |
| (D to P and A to P) | | |
| Residue 1, 2 and 3 | PPPFRHDSGYEVHH | 26 |
| (D to P and A to P | | |
| and E to P) | | |

7. Immunisation of Mice with TAP01_01, TAP01_02 and TAP01_4 and Effect on Plaque Load in 5XFAD Mice

5XFAD mice were treated between six weeks and 18 weeks of age with 10 mg/kg of the antibodies (TAP01_01, TAP01_02 and TAP01_4) i.p. Passive immunization with TAP01_4 (cloned as MoG1K) (also known as NT4X_S71H) antibody lowered plaque load for distinct Aβ species compared to an isotype control IgG1 antibody. TAP01_4 (MoG1K) significantly reduced plaques stained against pan-Aβ, pyroglutamate Aβ3-x, Thioflavin and TAP01.

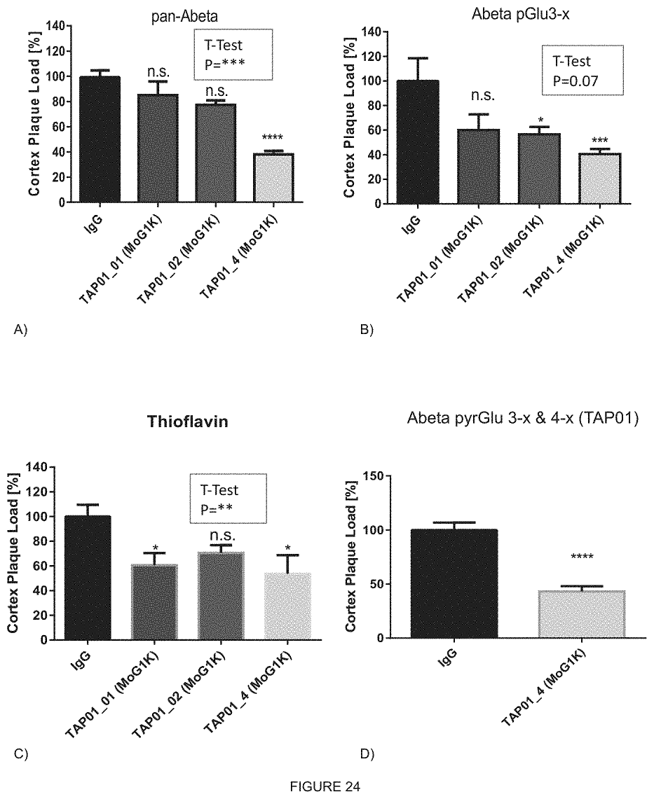

No effect was detected in pan-Aβ positive plaques for TAP01_01 (MoG1K) and a weak effect for TAP01_02 (MoG1K) as compared to the IgG control. TAP01_02 (MoG1K) significantly reduced plaques stained against, pyroglutamate Aβ3-x. The TAP01_01 (MoG1K) and TAP01_02 (MoG1K) treated group showed significantly reduced fibrillar Aβ deposits demonstrated by Thioflavin staining (FIG. 24)

8. Active Immunisation of Mice with Constrained Cyclic Peptide

5XFAD mice were immunized at 6 weeks of age for 12 weeks with antigen [Thioacetal bridged amyloid-beta peptide 1-14—KLH conjugate; DAC*FRHDSGYEC*HH[Cys]-amide (*S—CH—S bridged, cyclised via positions 3 and 12)] emulsified in complete Freund's adjuvant (CFA), followed by booster doses of protein emulsified in incomplete Freund's adjuvant (IFA). Mice were acclimated at our facility for at least 7 days before immunization. Mice were injected with antigen emulsified in CFA subcutaneously at two sites on the back, injecting 0.05 to 0.1 mL at each site (total of 0.1 to 0.2 mL per mouse).

Booster injections of antigen emulsified in IFA was administered at day 14, day 28, day 42 and 10 weeks after immunization with antigen/CFA emulsion. The booster is given as a single subcutaneous injection with 0.1 mL of IFA emulsion, at one site on the back. A serum sample was isolated from the mice after sacrifice of the mice (18 weeks of age), and antibody concentration was tested.

### 18F-FDG-PET/MRI Imaging

18F-FDG-PET/MRI was performed on 5xFAD mice as well as age matched C57Bl/6J wild type mice. Mice were fasted overnight and blood glucose levels were measured before tracer injection. 11.46 to 20.53 MBq (mean 16.81 MBq) 18F-FDG was injected intravenously into a tail vein with a maximum volume of 200 μl followed by an uptake period of 45 minutes. Mice were awake during the uptake process. PET scans were performed for 20 minutes using a small animal 1 Tesla nanoScan PET/MRI (Mediso, Hungary). Mice were anesthetized with isoflurane supplemented with oxygen during the scans and kept on a heated bed (37° C.). Respiratory rate was measured throughout the imaging process. MRI-based attenuation correction was conducted with the material map (matrix 144×144×163 with a voxel size of 0.5×0.5×0.6 mm³, TR: 15 ms, TE 2.032 ms and a flip angle of 25°) and the PET images were reconstructed using the following parameters: matrix 136×131×315, voxel size 0.23×0.3×0.3 mm³.

### 18F-Florbetaben-PET/MRI for Amyloid-Plaque Load

7.5-24 MBq (mean 14 MBq) of 18F-Florbetaben was administered intravenously into a tail vein with a maximum volume of 200 μl. After an uptake period of 40 minutes, mice were anesthetized and scanned as described above. PET acquisition time was 30 minutes. MRI-based attenuation correction was conducted with the material map (matrix 144×144×163 with a voxel size of 0.5×0.5×0.6 mm³, TR:15 ms, TE 2.032 ms and a flip angle of 25°), and the PET images were reconstructed with the following parameters: matrix 136×131×315 with a voxel size of 0.23×0.3×0.3 mm³ (Bouter et al, (2019), Frontiers in Aging Neuroscience vol. 10:425).

### Image Analysis

All images were analyzed using PMOD v3.9 (PMOD Technologies, Switzerland) as described before (Bouter et al). Briefly, a predefined MRI-based mouse brain atlas template was used to define different volumes of interest (VOIs) including whole brain volume as well as the amygdala, brain stem, cerebellum, cortex, hippocampus, hypothalamus, midbrain, olfactory bulb, septum/basal forebrain, striatum and thalamus. PET VOI statistics (kBq/cc) were generated for all brain areas and standardized uptake values (SUVs) were calculated [SUV=tissue activity concentration average (kBq/cc)×body weight (g)/injected dose (kBq)] for semi-quantitative analysis. SUVs of 18F-FDG-PET scans were corrected for measured blood glucose levels [SUVGIc=SUV x blood glucose level (mg/dl)]. SUVs of 18F-Florbetaben scans were further normalized by SUVs within the cerebellum VOI and obtained ratios (SUVr) were used for further analysis.

### Results

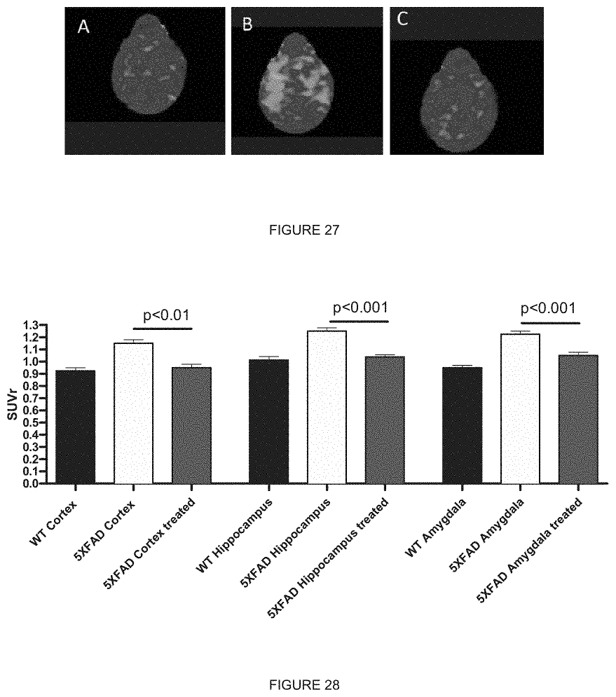

Amyloid-plaque imaging with the amyloid-plaque tracer fluorbetaben was performed in immunized 5XFAD (n=5), two 5XFAD mouse control and two wildtype mice (all female, age 4.5-5.5 months of age). The results are shown in FIGS. 27 and 28. None of the immunized 5XFAD mice showed retention of fluorbetaben in cortex, hippocampus and amygdala, which clearly demonstrates that the amyloid-plaque signal was drastically reduced. The cyclic peptide used for immunization is specific for an N-truncated amyloid-beta oligomer and antibodies induced by this cyclic peptide do not react with full-length amyloid-beta 1-42. The cyclic peptide (which is a mimic of the hairpin structure of the truncated peptide against which Abs are raised) used for immunization resulted in the clearance of amyloid-plaques in 5XFAD brain, which are mostly comprised of full-length amyloid-beta 1-42, only a minor fraction is N-truncated amyloid-beta. Therefore, this indicates that cyclic peptides raise antibodies that bind truncated amyloid-beta and these dissolve the plaques. The hairpin structure is the seeding factor for Alzheimer plaques and these can be removed by cyclic peptides active immunization.

### Summary

A novel epitope has been identified that the TAP01 and TAP01_01 antibodies bind to. These antibodies bind only the low molecular weight oligomers and not plaques as compared to several comparator antibodies which also bind plaques. Although a number of antibodies are able to bind to different regions of the amyloid peptide sequence, only the TAP01 is able to bind to the cyclic/hairpin conformation of the amyloid peptide. Therefore, the cyclic peptide generated which mimics the hairpin epitope of the Aβp3-42 could be used in active immunisation, to induce the generation of antibodies in a subject which are specific to low molecular weight oligomers.

### 9. Active Immunisation of Cyclised Aβ Peptides in 5XFAD Mice: Amyloid Load by Immunohistochemistry in Brain Sections, and In Vivo Glucose Metabolism by 18F-FDG-PET/MRI Imaging

5XFAD mice were immunized with antigen [Thioacetal bridged amyloid-beta peptide 1-14—KLH conjugate; DAC*FRHDSGYEC*HH[Cys]-amide (*S—CH—S bridged, cyclised via positions 3 and 12)] as described previously.

### In Vivo Imaging

In vivo glucose metabolism on Alzheimer mice (5XFAD) as well as age matched C57Bl/6J wild type mice was analysed by 18F-FDG-PET/MRI imaging as described above.

### Immunohistochemical Staining of Paraffin Sections

Mice were sacrificed via CO₂ anesthetization followed by cervical dislocation. Brain samples were carefully dissected and post-fixed in 4% phosphate-buffered formalin at 4° C. Human and mouse tissue samples were processed as previously described 4. In brief, 4 μm paraffin sections were deparaffinized in xylene, followed by rehydration in a series of ethanol. After H₂O₂ treatment to block endogenous peroxidases, sections were boiled in 0.01 M citrate buffer for antigen retrieval, followed by three min incubation in 88% formic acid. Non-specific binding sites were blocked by treatment with skim milk and fetal calf serum in PBS, prior to the addition of the primary antibodies. The following antibodies were used: polyclonal antibody 24311 against pan-A135, monoclonal antibody 1-57 against pyroglumatate Aβ3-X, (Synaptic Sytems, GOttingen, Germany; 1 mg/ml; 1:500), and TAP01_4 (1:200; 2 mg/ml). Corresponding biotinylated secondary anti-human and anti-mouse antibodies (1:200) were purchased from DAKO (Glostrup, Denmark). Staining was visualized using the ABC method, with a Vectastain kit (Vector Laboratories, Burlingame, USA) and diaminobenzidine (DAB) as chromogen. Counterstaining was carried out with hematoxylin.

### Quantification of Abeta Load

Plaque load was quantified as previously described (G. Antonios et al., Alzheimer therapy with an antibody against N-terminal Abeta 4-X and pyroglutamate Abeta 3-X. Scientific reports 5, 17338 (2015)). 5-6 paraffin embedded sections, which were at least 80 μm afar from each other, were stained simultaneously with DAB as chromogen. For Thioflavin S fluorescent staining tissue sections were deparaffinized and rehydrated, washed twice in deionized water and then treated with 1% (w/v) ThioflavinS in aqueous solution and counterstained in a 1% (w/v) aqueous solution of 4′6-diamidin-2-phenylindol. The relative Aβ load was evaluated using an Olympus BX-51 microscope equipped with an Olympus DP-50 camera and the ImageJ software (NIH, USA). Representative pictures of 100× magnification were systematically captured. Using ImageJ the pictures were binarized to 8-bit black and white pictures and a fixed intensity threshold was applied defining the DAB staining. Measurements were performed for a percentage area covered by DAB staining, as well as for the number of grains per mm² and the average size of the grains.

### Results

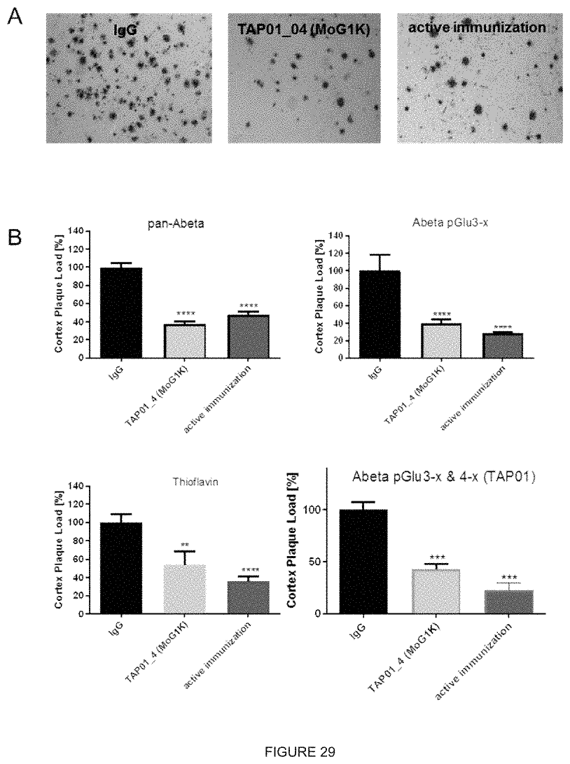

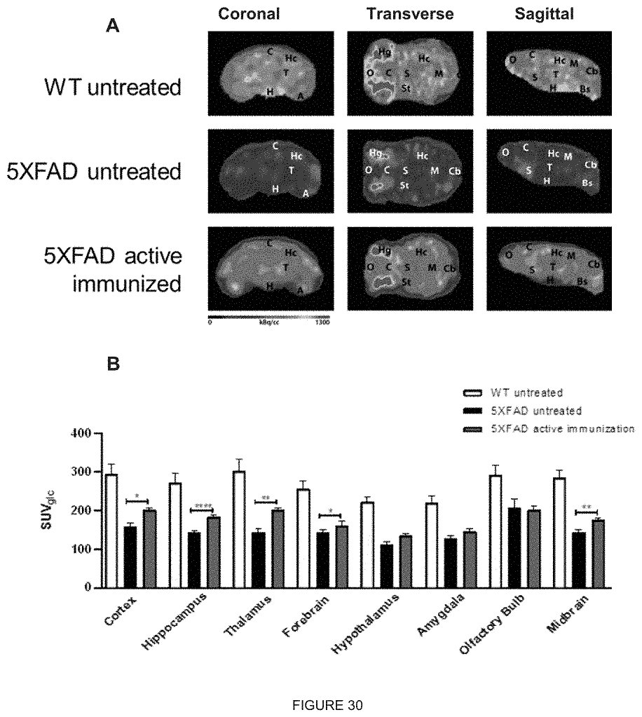

We assessed the effect of active immunisation on plaque load in brain sections (FIG. 29), and in vivo glucose metabolism using PET/MRI imaging (FIG. 30).

Active immunization of the AD mouse model 5XFAD with the 3-12 linked Aβ1-14 cyclic peptide resulted in a reduction in amyloid-plaque load in brain tissue.

The immunostaining of the plaque load in the cortex of 5XFAD mice treated with TAP01_04 antibody or active immunization with cyclized Aβ peptide (FIG. 29) was in good agreement with the florbetaben retention signals seen for cortex, hippocampus and amygdala shown in FIG. 28. 5XFAD mouse cortical sections were stained with antibodies against pan-Abeta, pyroglutamate Aβ3-X, ThioflavinS and Aβ4-X. 5XFAD mice with active immunization showed a significant reduction in plaque load with antibody stainings and ThioflavinS staining (FIG. 29).

Glucose uptake was assessed by 18F-FDG imaging in WT and 5XFAD mice. The rescue of the glucose uptake signals was seen in cortex, hippocampus, thalamus, forebrain and the midbrain in 5XFAD mice with active immunization (FIG. 30B).

### 10. Active Immunisation of Cyclised Aβ Peptides in Tg4-42 Mice and Treatment Effect on Hippocampus Function

Tg4-42 mice were immunized at 6 weeks of age for 12 weeks with antigen [Thioacetal bridged Abeta peptide 1-14—KLH conjugate; DAC*FRHDSGYEC*HH[Cys]-amide (*S—CH—S bridged)] emulsified in complete Freund's adjuvant (CFA), followed by booster doses of protein emulsified in incomplete Freund's adjuvant (IFA). Booster injections of antigen emulsified in IFA was administered at day 14, day 28, day 42, thereafter monthly (3 times after month 4, 5 and 6), after immunization with antigen/CFA emulsion. Mice were acclimated at our facility for at least 7 days before immunization. Mice were injected with antigen emulsified in CFA or IFA subcutaneously at two sites on the back, injecting 0.05 to 0.1 mL at each site (total of 0.1 to 0.2 mL per mouse). The booster was given as a single subcutaneous injection with 0.1 mL of IFA emulsion, at one site on the back. A serum sample was isolated from the mice after sacrifice of the mice for titer determination.

### Spatial Reference Memory by Morris Water Maze

Spatial reference memory in mice was evaluated using the Morris water maze (R. Morris, Developments of a water-maze procedure for studying spatial learning in the rat. J. Neurosci. Methods 11, 47-60 (1984)) as described previously (Y. Bouter et al., N-truncated amyloid beta (Abeta) 4-42 forms stable aggregates and induces acute and long-lasting behavioral deficits. Acta Neuropathol 126, 189-205 (2013)).

### Quantification of Neuron Numbers Using Unbiased Stereology

Stereological analysis was performed as previously described (G. Antonios et al., Alzheimer therapy with an antibody against N-terminal Abeta 4-X and pyroglutamate Abeta 3-X. Scientific reports 5, 17338 (2015)). The hippocampal cell layer CA1 (Bregma −1.22 to −3.52 mm) was delineated on cresyl violet-stained sections and analysed with a stereology workstation (Olympus BX51 with a motorized specimen stage for automatic sampling), StereoInvestigator 7 (MicroBrightField, Williston, USA) and a 100x oil lens (NA=1.35).

### Results

Active immunization of the AD mouse model Tg4-42 with 3-12 linked A61-14 cyclic peptide, which revealed a substantial rescue of learning and memory deficits, together with significantly reduced loss of neurons.

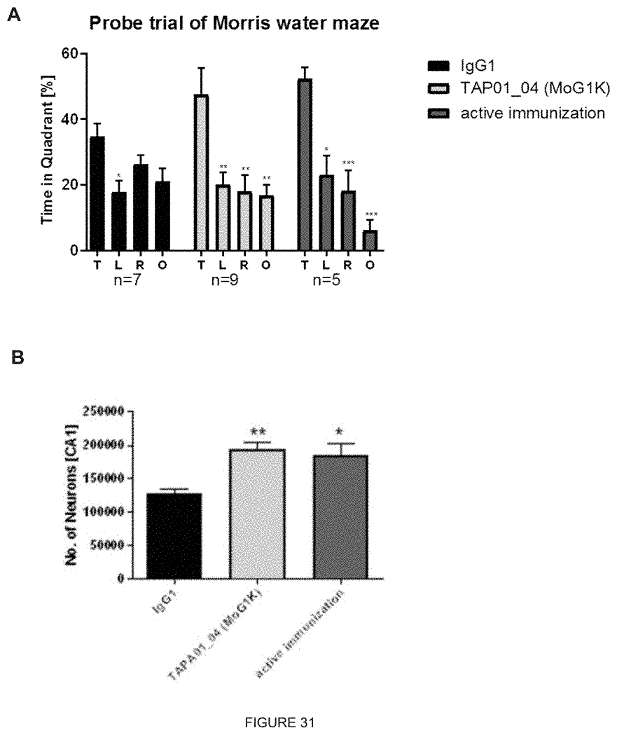

The treatment effect of active immunization was assessed in 6.5 month old Tg4-42 mice on hippocampus-dependent learning and memory by the Morris water maze test (FIG. 31A) and by counting the total number of CA1 neurons in the hippocampus (FIG. 31B). This was compared to effects of passive immunization with TAP01_04 and IgG1 control antibodies. Active immunization as well as passive immunisation with TAP01_04 antibody significantly improved spatial reference memory defects and the number of CA1 neurons in aged Tg4-42 having received active or TAP01_04 immunization.

### 11. Active Immunisation of Cyclised Aβ Peptides as a Potential Vaccine Approach for the Treatment of AD

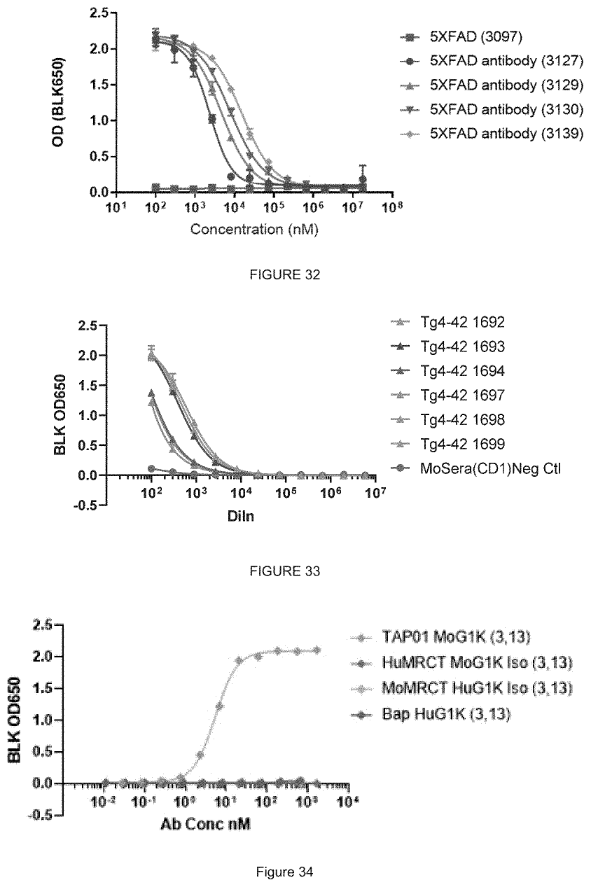

Animals (SXFAD and Tg4-42 mice) were immunised with thioacetal bridged cyclic peptide 1-14 and sera were screened for binding to biotinylated cyclised peptide as previously described above. All mice generated a good immune response (FIGS. 32 and 33).

### Summary

The therapeutic potential of active immunisation for the treatment and prevention of Alzheimer's Disease with a cyclic peptide which mimics the hairpin epitope of the Aβp3-42 has been shown by the above results.

1. A cyclic peptide comprising an amino acid sequence having the structure of formula (I) and variants thereof:
X₁X₂X₃FX₄HDSGX₅X₆X₇X₈H (I)
wherein:
X₁ is absent or any amino acid; and
X₂ is alanine or cysteine;
X₃ is glutamic acid or cysteine;
X₄ is arginine or cysteine;
X₅ is tyrosine or cysteine;
X₆ is glutamic acid or cysteine;
X₇ is valine or cysteine; and
X₈ is histidine or cysteine
wherein only one of X₁, X₂, X₃ and X₄ is cysteine and wherein only one of X₅, X₆, X₇ and X₈ is cysteine and the peptide is cyclized via the cysteine residue position at 1, 2, 3, or 5 and the cysteine residue at position 10, 11, 12 or 13.

2. A cyclic peptide according to claim 1 and variants thereof wherein:
X₁ is absence or any amino acid; and
wherein:
a) X₁ is cysteine, X₂ is alanine, X₃ is glutamic acid, X₄ is arginine, X₅ is tyrosine, X₆ is glutamic acid, X₇ is histidine and X₈ is cysteine;
b) X₂ is alanine, X₃ is cysteine, X₄ is arginine, X₅ is tyrosine, X₆ is glutamic acid, X₇ is cysteine and X₈ is histidine;
c) X₂ is cysteine, X₃ is glutamic acid, X₄ is arginine, X₅ is tyrosine, X₆ is glutamic acid, X₇ is cysteine and X₈ is histidine;
d) X₂ is cysteine, X₃ is glutamic acid, X₄ is arginine, X₅ is cysteine, X₆ is glutamic acid, X₇ is valine and X₈ is histidine;
e) X₂ is cysteine, X₃ is glutamic acid, X₄ is arginine, X₅ is tyrosine, X₆ is glutamic acid, X₇ is valine and X₈ is cysteine;
f) X₂ is cysteine, X₃ is glutamic acid, X₄ is arginine, X₅ is tyrosine, X₆ is cysteine, X₇ is valine and X₈ is histidine;
g) X₂ is alanine, X₃ is cysteine, X₄ is arginine, X₅ is tyrosine, X₆ is cysteine, X₇ is valine and X₈ is histidine;
h) X₂ is alanine, X₃ is glutamic acid, X₄ is cysteine, X₅ is tyrosine, X₆ is glutamic acid, X₇ is cysteine and X₈ is histidine;
i) X₂ is alanine, X₃ is cysteine, X₄ is arginine, X₅ is cysteine, X₆ is glutamic acid, X₇ is histidine and X₈ is histidine; or
j) X₂ is alanine, X₃ is cysteine, X₄ is arginine, X₅ is tyrosine, X₆ is glutamic acid, X₇ is histidine and X₈ is cysteine;
wherein the peptide is cyclized via the two cysteine residues.

3. A cyclic peptide according to claim 1 or 2 comprising an amino acid sequence or variant thereof selected from:
a) DACFRHDSGYECHH wherein the peptide is a cyclized via the cysteine residues located at positions 3 and 12;
b) DACFRHDSGYEVCH wherein the peptide is a cyclized via the cysteine residues located at positions 3 and 13.
c) CAECFRHDSGYEVCH wherein the peptide is a cyclized via the cysteine residues located at positions 1 and 13;
d) DCEFRHDSGYECHH wherein the peptide is a cyclized via the cysteine residues located at positions 2 and 12;
e) DCEFRHDSGCEVHH wherein the peptide is a cyclized via the cysteine residues located at positions 2 and 10;
f) DCEFRHDSGYEVCH wherein the peptide is a cyclized via the cysteine residues located at positions 2 and 13;
g) DCEFRHDSGYCVHH wherein the peptide is a cyclized via the cysteine residues located at positions 2 and 11;
h) DACFRHDSGYCVHH wherein the peptide is a cyclized via the cysteine residues located at positions 3 and 11;
i) DAEFCHDSGYECHH wherein the peptide is a cyclized via the cysteine residues located at positions 5 and 12; and
j) DACFRHDSGCEVHH wherein the peptide is a cyclized via the cysteine residues located at positions 3 and 10.

4. A cyclic peptide according to any one of claim 1 or 2 wherein X₁ is proline or aspartic acid.

5. A cyclic peptide according to any one of claims 1 to 4 wherein the peptide is cyclized via a bridge connecting the two cysteine residues.

6. A cyclic peptide according to any one of claims 1 to 5 wherein the peptide is cyclized via a bridge having the formula —S—S— or —S—CH₂—S— between the two cysteine residues.

7. A cyclic peptide according to any one of claims 1 to 6 comprising:
a) the amino acid sequence DACFRHDSGYECHH or variant thereof wherein the peptide is cyclized via the cysteine residues located at positions 3 and 12; or
b) the amino acid sequence DACFRHDSGYEVCH or variant thereof wherein the peptide is cyclized via the cysteine residues located at positions 3 and 13; or
c) the amino acid sequence CAECFRHDSGYEVCH or variant thereof wherein the peptide is a cyclized via the cysteine residues located at positions 1 and 13

8. A cyclic peptide according to any one of claims 1 to 7 comprising:
a) the amino acid sequence DACFRHDSGYECHH or variant thereof wherein the peptide is a cyclic peptide formed by the bridge having the formula —S—CH₂—S— between the two cysteine residues at positions 3 and 12; or
b) the amino acid sequence DACFRHDSGYEVCH or variant thereof wherein the peptide is a cyclic peptide formed by the bridge having the formula —S—CH₂—S— between the two cysteine residues at positions 3 and 13; or
c) the amino acid sequence CAEFRHDSGYEVCH or variant thereof wherein the peptide is a cyclic peptide formed by the bridge having the formula —S—CH₂—S— between the two cysteine residues at positions 1 and 13.

9. A cyclic peptide according to any one of claims 1 to 8 consisting of:
a) the amino acid sequence DACFRHDSGYECHH or variant thereof wherein the peptide is a cyclic peptide formed by the bridge having the formula —S—CH₂—S— between the two cysteine residues at positions 3 and 12; or
b) the amino acid sequence DACFRHDSGYEVCH or variant thereof wherein the peptide is a cyclic peptide formed by the bridge having the formula —S—CH₂—S— between the two cysteine residues at positions 3 and 13; or
c) the amino acid sequence CAEFRHDSGYEVCH or variant thereof wherein the peptide is a cyclic peptide formed by the bridge having the formula —S—CH₂—S— between the two cysteine residues at positions 1 and 13.

10. A cyclic peptide comprising an amino acid sequence having at least 85% sequence identity with:
a) the amino acid sequence DACFRHDSGYECHH or variant thereof wherein the peptide comprises the cysteine residues at positions 3 and 12 via which the peptide is cyclised and the phenylalanine residue at position 4; or
b) the amino acid sequence DACFRHDSGYEVCH or variant thereof wherein the peptide comprises the cysteine residues at positions 3 and 13 via which the peptide is cyclised and the phenylalanine residue at position 4; or
c) the amino acid sequence CAEFRHDSGYEVCH or variant thereof wherein the peptide comprises the cysteine residues at positions 1 and 13 via which the peptide is cyclised and the phenylalanine residue at position 4.

11. A cyclic peptide according to any one of claims 1 to 10 comprising the amino acid sequence DACFRHDSGYECHH or variant thereof wherein the peptide is a cyclic peptide formed by the bridge having the formula —S—CH₂—S— between the cysteine residues at positions 3 and 12.

12. A pharmaceutical composition comprising the cyclic peptide according to any one of claims 1 to 11 and a pharmaceutically acceptable carrier.

13. A pharmaceutical composition according to claim 12 further comprising an adjuvant.

14. A method of treating a neurodegenerative disease comprising administering a cyclic peptide according to any one of claims 1 to 11 or a composition according to any one of claim 12 or 13 to an individual in need thereof.

15. A method according to claim 14 wherein the neurodegenerative disease is Alzheimer's disease.

16. A method for inducing an immune response in a subject comprising administering a cyclic peptide according to any one of claims 1 to 11 or a composition according to any one of claim 12 or 13 to the subject

17. A method according to claim 16 wherein the immune response produces antibodies against amyloid beta in the form of low molecular weight amyloid-beta oligomers.

18. A cyclic polypeptide according to any one of claims 1 to 11 to for use in treating a neurodegenerative disease.

19. A cyclic peptide for use according to claim 18 wherein the neurodegenerative disease is Alzheimer's disease.

20. A method of producing a cyclic peptide according to any one of claims 1 to 11 comprising the steps of:
(a) synthesizing a linear peptide comprising the sequence of the peptide as defined in any one of claims 1 to 11; and
(b) cyclizing the linear peptide via the cysteine residue to obtain the cyclic peptide according to any one of claims 1 to 11.

21. A method for the generating an antibody that specifically recognizes low molecular weight oligomers of amyloid beta comprising:
(a) immunizing an animal with a cyclic peptide or variant thereof according to any of claims 1-11; and
(b) obtaining the antibodies generated by the immunization in step (a).


SEQUENCE LISTING (26 sequences)

SEQ ID NO: 1 - protein, 14 aa, Artificial sequence
  Formula (I)
  MISC_FEATURE (1)..(1): Xaa = absent or any amino acid
  MISC_FEATURE (2)..(2): Xaa = A or C
  MISC_FEATURE (3)..(3): Xaa = E or C
  MISC_FEATURE (5)..(5): Xaa = R or C
  MISC_FEATURE (10)..(10): Xaa = Y or C
  MISC_FEATURE (11)..(11): Xaa = E or C
  MISC_FEATURE (12)..(12): Xaa = V or C
  MISC_FEATURE (13)..(13): Xaa = H or C
XXXFXHDSGXXXXH

SEQ ID NO: 2 - protein, 14 aa, Artificial sequence
  Formula (II)
  MISC_FEATURE (1)..(1): Xaa = absent or any amino acid
XACFRHDSGYECHH

SEQ ID NO: 3 - protein, 14 aa, Artificial sequence
  Original
DAEFRHDSGYEVHH

SEQ ID NO: 4 - protein, 14 aa, Artificial sequence
  3,12
DACFRHDSGYECHH

SEQ ID NO: 5 - protein, 14 aa, Artificial sequence
  2,12
DCEFRHDSGYECHH

SEQ ID NO: 6 - protein, 14 aa, Artificial sequence
  4,12
DAECRHDSGYECHH

SEQ ID NO: 7 - protein, 14 aa, Artificial sequence
  5,12
DAEFCHDSGYECHH

SEQ ID NO: 8 - protein, 14 aa, Artificial sequence
  3,11
DACFRHDSGYCVHH

SEQ ID NO: 9 - protein, 14 aa, Artificial sequence
  2,11
DCEFRHDSGYCVHH

SEQ ID NO: 10 - protein, 14 aa, Artificial sequence
  2,10
DCEFRHDSGCEVHH

SEQ ID NO: 11 - protein, 14 aa, Artificial sequence
  3,10
DACFRHDSGCEVHH

SEQ ID NO: 12 - protein, 14 aa, Artificial sequence
  2,13
DCEFRHDSGYEVCH

SEQ ID NO: 13 - protein, 14 aa, Artificial sequence
  3,13
DACFRHDSGYEVCH

SEQ ID NO: 14 - protein, 14 aa, Artificial sequence
  1,13
CAEFRHDSGYEVCH

SEQ ID NO: 15 - protein, 14 aa, Artificial sequence
  1,12
CAEFRHDSGYECHH

SEQ ID NO: 16 - protein, 14 aa, Artificial sequence
  1,11
CAEFRHDSGYCVHH

SEQ ID NO: 17 - protein, 14 aa, Artificial sequence
  1,10
CAEFRHDSGCEVHH

SEQ ID NO: 18 - protein, 42 aa, Homo sapiens [sequence omitted]

SEQ ID NO: 19 - protein, 40 aa, Homo sapiens
DAEFRHDSGYEVHHQKLVFFAEDVGSNKGAIIGLMVGGVV

SEQ ID NO: 20 - protein, 40 aa, Homo sapiens
EFRHDSGYEVHHQKLVFFAEDVGSNKGAIIGLMVGGVVIA

SEQ ID NO: 21 - protein, 39 aa, Homo sapiens
FRHDSGYEVHHQKLVFFAEDVGSNKGAIIGLMVGGVVIA

SEQ ID NO: 22 - protein, 14 aa, Artificial sequence
  Residue 1 (D to P)
PAEFRHDSGYEVHH

SEQ ID NO: 23 - protein, 14 aa, Artificial sequence
  Residue 2 (A to P)
DPEFRHDSGYEVHH

SEQ ID NO: 24 - protein, 14 aa, Artificial sequence
  Residue 3 (E to P)
DAPFRHDSGYEVHH

SEQ ID NO: 25 - protein, 14 aa, Artificial sequence
  Residue 1 and 2 (D to P and A to P)
PPEFRHDSGYEVHH

SEQ ID NO: 26 - protein, 14 aa, Artificial sequence
  Residue 1, 2 and 3 (D to P and A to P and E to P)
PPPFRHDSGYEVHH